(19) United States
(12) Patent Application Publication
(10) Pub. No.: US 2022/0313972 A1
(43) Pub. Date: Oct. 6, 2022

(54) TOGGLING VASCULAR ACCESS PORT

(71) Applicant: Portal Access, Inc., Wilmington, DE (US)

(72) Inventor: Michael Gabriel TAL, Tel Aviv (IL)

(21) Appl. No.: 17/625,637

(22) PCT Filed: Jul. 8, 2020
(86) PCT No.: PCT/US2020/041140

§ 371 Date: Jan. 7, 2022

Related U.S. Application Data
(63) Continuation of application No. PCT/US2020/020759, filed on Jan. 15, 2020.
(60) Provisional application No. 62/872,022, filed on Jul. 9, 2019.
(60) Provisional application No. 62/961,374, filed on Jan. 15, 2020.

(51) Int. Cl. A61M 39/02

(52) U.S. Cl. CPC A61M 39/0208

(57) ABSTRACT

Disclosed are toggling vascular access port and methods of deploying thereof. The port includes a port body coupled with a septum member covering a cavity defined by the port body, and at least one port body extension restrictedly movable along an at least one defined route on the port body. The port is selectively changeable from a delivery configuration to a deployed configuration by moving the at least one port body extension along the at least one defined route wherein the at least one port body extension and the port body are approximated along a median plane of the port body and laterally opposing portions of the at least one port body extension are parted transversely to the median plane, thereby reducing length-to-width ratio of the toggling vascular access port.

### RELATED APPLICATIONS

This application claims priority to U.S. Provisional Patent Application No. 62/872,022, filed on Jul. 9, 2019, to U.S. Provisional Patent Application No. 62/961,374, filed on Jan. 15, 2020, and to PCT Application No. PCT/US20/20759, filed on Mar. 3, 2020; The entire disclosures of all the related applications set forth in this section are hereby incorporated by reference in their entireties.

### BACKGROUND

Repeated needle pricking for facilitating delivery or withdrawal of fluids (e.g., medication or agents) to patient's vascular system causes harm to local tissues and decreases target blood vessel functionality and needle placement accuracy. This phenomenon is often evident in chronic diabetes, dialysis or chemotherapy patients, for example, who require continuous and repeated intravenous fluids administration for prolonged periods.

A vascular access port is a device that enables such repeated pricking and fluid administration while minimizing the accumulated harm caused by needle pricking and powered injections of fluid. The access port is subcutaneously implanted, in a surgically formed pocket in proximity to a large blood vessel, usually in the chest. It is basically formed of a port body enclosing a cavity, which is capped with a septum member configured for supporting the upper skin layers and for accepting repeated needle pricking therethrough for intravascular fluid deliveries sealed to the surrounding body tissues. The port is attached to a catheter (a thin, flexible tube) which provides fluid communication with a large blood vessel, such as the superior vena cava, in order to allow the injected fluid to dilute in the blood stream.

The implantation of a port is considered a minor procedure performed under local or general anesthesia by an interventional radiologist or surgeon. First, the surgeon achieve access to the desired vein, a skin incision is made afterwards in the access point. Second larger incision is made above the desired location of the port, through which a pocket-like subcutaneous void is made using blunt device. The catheter is extended subcutaneously between the two incisions using a blunt tunneler. One end of the catheter is then inserted into the vein and its other end is coupled to the port. Optionally, during deployment the catheter is cut to a desired length.

Besides progress made in past year in access ports design, there is still a need to develop ports and methods of implantation and deployment thereof, which are less traumatic and invasive, and simpler to perform, potentially also by non-surgical medical personnel.

It should be noted that this Background is not intended to be an aid in determining the scope of the claimed subject matter nor be viewed as limiting the claimed subject matter to implementations that solve any or all of the disadvantages or problems presented above. The discussion of any technology, documents, or references in this Background section should not be interpreted as an admission that the material described is prior art to any of the subject matter claimed herein.

### SUMMARY

The present disclosure, in some embodiments thereof, relates to devices and methods for facilitating and/or improving repeated deliveries of fluids (e.g., fluids carrying nutrients, medicament and/or agents such as chemotherapy agents) into vasculature of a subject, and more particularly, but not exclusively, to vascular access ports and methods of delivery and deployment thereof in a body of a subject.

In certain embodiments, there is provided a toggling vascular access port, comprising:

a port body coupled with a septum member covering a cavity defined by the port body; and at least one port body extension restrictedly movable along an at least one defined route on the port body.

In some embodiments, the toggling vascular access port is selectively changeable from a delivery configuration to a deployed configuration by moving the at least one port body extension along the at least one defined route wherein the at least one port body extension and the port body are approximated along a median plane of the port body and laterally opposing portions of the at least one port body extension are parted transversely to the median plane, thereby reducing length-to-width ratio of the toggling vascular access port.

In some embodiments, when in the delivery configuration, front portion of the at least one port body extension is positioned axially distally to the port body.

In some embodiments, when in the deployed configuration, the at least one port body extension is fixedly connected to the port body, and is optionally releasably connected thereto for allowing selective reverting from the deployed configuration to the delivery configuration.

In some embodiments, the at least one port body extension includes a first arm located right to the median plane and a second arm located left to the median plane.

In some embodiments, when in the delivery configuration, each one of the first and second arms includes a wide head portion and a narrow body portion, the head portions and in contact with each other along the median plane and the body portions surround distal portion of the port body from both sides of the median plane.

In some embodiments, the toggling vascular access port is configured to separate between the head portions transversely to the median plane when the toggling vascular access port shifts from the delivery configuration.

In some embodiments, when the toggling vascular access port is in the deployed configuration, the head portions are in juxtaposition from both sides of the port body with each one of the head portions forming a gap with a front end of the port body, thereby allowing tissue ingrowth therebetween.

In some embodiments, the port body has an inferior portion and a posterior portion, the posterior portion is connected to the septum member and the inferior portion surrounds the cavity below the septum member and includes a first lateral surface spanning most or all right side of the inferior portion and a second lateral surface spanning most or all left side of the inferior portion.

In some embodiments, the inferior portion is oval, elliptical, subelliptical, pyriform or vesica piscis shaped along the first and second lateral surfaces.

In some embodiments, each one of the first and second lateral surfaces is curved and has a constant radius of curvature being substantially greater than distance between opposing vertexes of the first and second lateral surfaces.

In some embodiments, the posterior portion includes a brim-like rim projecting laterally outwardly above the first and second lateral surfaces, configured to cover a seam line formed between the port body and the port body extension when in the deployed configuration.

In some embodiments, the at least one port body extension comprising a first arm slidably connected to the first lateral surface and restrictedly movable along a first defined route and a second arm slidably connected to the second lateral surface and restrictedly movable along a second defined route.

In some embodiments, each one of the right and left sides of the inferior portion and/or of the at least one port body extension includes a rail mechanism along a length of the respective first or second lateral surface, facilitating the first and second defined routes, respectively.

In some embodiments, each rail mechanism includes a pair of geometrically mating curved elongated ridge and groove longitudinally interengaging with each other, wherein one of the ridge and the groove extend along the respective first or second lateral surface and the other of the ridge and the groove extend along the respective first or second arm.

In some embodiments, when the toggling vascular access port is in the deployed configuration, the first arm covers the first lateral surface and the second arm covers the second lateral surface.

In some embodiments, the toggling vascular access port is configured such that inner surface of each respective first or second arm is in contact with the respective first or second lateral surface with substantially no gap therebetween.

In some embodiments, when changing from the delivery configuration to the deployed configuration, the at least one port body extension rotates around an axis of rotation and slides with an inner surface thereof on at least one of two opposing sides of an inferior portion of the port body.

In some embodiments, the axis of rotation is located farther than and beyond a closer one of the two opposing sides of the inferior portion relative to the inner surface of the port body extension.

In some embodiments, the inner surface of the at least one port body extension is configured to cover a front end of the port body and not cover most of the at least one of the two opposing sides of the inferior portion, when the toggling vascular access port is in the delivery configuration.

In some embodiments, when the toggling vascular access port is in the deployed configuration, the at least one port body extension covers at least most of the two opposing sides of the port body inferior portion.

In some embodiments, total volume of the at least one port body extension is at least 15% of total volume of the port body.

In some embodiments, the port body is configured to move distally towards the at least one port body extension when the toggling vascular access port changes to the deployed configuration.

In some embodiments, the port body is configured to interlock with the at least one port body extension when the toggling vascular access port is in the deployed configuration.

In some embodiments, the at least one port body extension includes a front edge pointing distally away from the port body and configured to cause atraumatic separation of tissue layers when forced to pass therebetween.

In some embodiments, the port body and/or the at least one port body extension is configured to maintain size and shape thereof when the toggling vascular access port changes to the deployed configuration.

In some embodiments, the at least one port body extension is configured to stabilize and/or fixate the port body in-place in a target implantation site in the subject body when the toggling vascular access port changes to the deployed configuration.

In some embodiments, the at least one port body extension is configured for insertion into a subcutaneous void before and/or separately from the port body when the toggling vascular access port is in the delivery configuration, and to force increase is volume enclosed by the subcutaneous void by expanding therein when the toggling vascular access port changes to the deployed configuration.

In some embodiments, when the toggling vascular access port is in the deployed configuration, is generally triangular shaped.

In some embodiments, the septum member is generally elliptical.

In some embodiments, when the toggling vascular access port is in the deployed configuration, a rear end of the port body is not covered with the port body extension.

In some embodiments, the port body rear end is coupled to a catheter connector configured for connecting to a proximal end of a catheter for facilitating fluid communication between the cavity and a lumen of the catheter.

In some embodiments, the toggling vascular access port comprising a port clamping portion located superiorly to the catheter connector.

In some embodiments, the port clamping portion includes a thin wall portion comprising opposing lateral surfaces extending parallel to the median plane from both sides thereof, the wall portion is configured for grasping and/or clamping by medical forceps including but not limited to Kelly forceps.

In some embodiments, the wall portion is configured as a septum dividing cavities formed in the rear end of the port body, the cavities are shaped and sized to accommodate a pair of tips of the medical forceps and to allow closing motion of the pair of tips in the cavities towards the wall portion and grasping of the wall portion with the pair of tips from both sides thereof.

In some embodiments, the at least one port body extension is rotatably and slidably connected to the port body and is configured to rotate around an axis of rotation and slide on an at least one of two opposing sides of the port body when changing from the delivery configuration to the deployed configuration.

In some embodiments, the toggling vascular access port further comprising a toggle anchoring mechanism integrated in the port body and/or the at least one port body extension and configured to rotate the at least one port body extension around an axis of rotation and translate the at least one port body extension along at least one of opposing sides of the port body when changing from the delivery configuration to the deployed configuration.

In some embodiments, the toggling vascular access port is configured such that the at least one defined route is parallel to a longitudinal axis of the port body so as to maintain a fixed distance between bottom of the port body and bottom of the at least one port body extension, when changing from the delivery configuration to the deployed configuration.

In some embodiments, a planar bottom surface of the port body and a planar bottom surface of the at least one port body extension are substantially coplanar when in the delivery configuration and/or when in the deployed configuration.

In some embodiments, the toggling vascular access port is configured such that the at least one defined route is inclined to a longitudinal axis of the port body so as to gradually increase or decrease distance between bottom of the port body and bottom of the at least one port body extension, when changing from the delivery configuration to the deployed configuration.

In some embodiments, a planar bottom surface of the port body and a planar bottom surface of the at least one port body extension diverge from being substantially coplanar when changing from the delivery configuration to the deployed configuration.

In some embodiments, a planar bottom surface of the port body and a planar bottom surface of the at least one port body extension remain substantially parallel to each other when changing from the delivery configuration to the deployed configuration.

In certain embodiments, there is provided a method for deploying a toggling vascular access port in a body of a subject. In some embodiments the method comprising at least one of the following steps (not necessarily in same order):

forming a surgical opening through skin tissue layers to a subcutaneous target implantation site in the subject body;

through the surgical opening, inserting the toggling vascular access port in a delivery configuration into the target implantation site, wherein the toggling vascular access port includes a port body and at least one port body extension; and

changing the toggling vascular access port into a deployed configuration by approximating the at least one port body extension and the port body along a median plane of the port body and parting laterally opposing portions of the at least one port body extension transversely to the median plane, thereby reducing length-to-width ratio of the toggling vascular access port, for anchoring the toggling vascular access port in the target implantation site.

In some embodiments, the changing causes pressing against surrounding tissues generally away from the median plane.

In some embodiments, the method further comprising:

creating a subcutaneous surgical tunnel between the surgical opening and the target implantation site; and

delivering the toggling vascular access port through the surgical tunnel to the target implantation site.

In some embodiments, the port body is coupled with a septum member covering a cavity defined by the port body.

In some embodiments, the port body has an inferior portion and a posterior portion, the posterior portion is connected to the septum member and the inferior portion surrounds the cavity below the septum member and includes a first lateral surface spanning most or all right side of the inferior portion and a second lateral surface spanning most or all left side of the inferior portion, wherein the at least one port body extension comprising a first arm slidably connected to the first lateral surface and a second arm slidably connected to the second lateral surface.

In some embodiments, the changing includes covering the first lateral surface with the first arm and the second lateral surface with the second arm such that inner surface of each respective first or second arm is in contact with the respective first or second lateral surface with substantially no gap therebetween.

In some embodiments, the at least one port body extension is restrictedly movable along an at least one defined route on the port body, and the changing includes sliding the at least one port body extension along the at least one defined route.

In some embodiments, the at least one defined route is inclined to a longitudinal axis of the port body, and the changing includes elevating the port body relative to the at least one port body extension.

In some embodiments, the elevating includes gradually increasing distance between bottom of the port body and bottom of the at least one port body extension.

In some embodiments, the changing includes pushing the port body distally relative to the at least one port body extension.

In some embodiments, a rear end of the port body comprising a port clamping portion, wherein the inserting includes clamping the port clamping portion with medical forceps and pushing the toggling vascular access port to the target implantation site with the medical forceps.

In some embodiments, the changing includes pulling the at least one port body extension while resisting motion of the port body using the forceps.

In some embodiments, the port clamping portion is located superiorly to a catheter connector coupled to the rear end of the port body, the method comprising connecting a proximal end of a catheter to the catheter connector for facilitating fluid communication between the cavity and a lumen of the catheter.

In certain embodiments, there is provided a method for implanting a vascular access port in a subject's body. In some embodiments, the method comprising at least one of the following steps (not necessarily in same order):

forming a first incision to a skin layer of the subject's body;

passing an elongated introducer, subcutaneously, from an entry point to the first incision, such that a distal end of the introducer emerges from within the skin layer proximately to the first incision;

coupling the vascular access port to the introducer;

withdrawing the vascular access port via the first incision until the vascular access port is fully implanted in a subcutaneous passage proximately to the entry point; and

uncoupling the introducer from the vascular access port.

In some embodiments, the method further comprising:

creating subcutaneous passage from the entry point to the first incision.

In some embodiments, the subcutaneous passage is created or enlarged by way of the passing or the withdrawing.

In some embodiments, the method further comprising:

forming a second incision to the skin, wherein the second incision includes the entry point or is formed across or proximately to the entry point.

In some embodiments, a distance from the entry point to the first incision is substantially greater than maximal dimension of the vascular access port, optionally particularly equal to or greater than 2 cm. In some embodiments, the introducer distal end is covered with a cover, and the connecting follows uncovering the introducer distal end. Optionally, the cover has a pointed or rounded front, wherein the subcutaneous passage is formed with the cover front by pushing the cover beneath the skin layer from the entry point to the first incision.

In some embodiments, the introducer includes a tubular body. In some such embodiments, the step of coupling the vascular access port to the introducer includes:

protruding a distal end of a shaft from within the tubular body; and

fastening to the vascular access port a shaft fastener provided at the shaft distal end.

In some embodiments, the step of coupling the vascular access port to the introducer follows at least one of the following steps:

delivering the shaft distal end through the tubular body; and

fixating the shaft to the introducer.

In some embodiments, the method further comprising:

connecting the vascular access port to a port body extension to form a unified structure having a final shape and size.

In some embodiments, the step of withdrawing the vascular access port includes:

positioning the port body extension proximately to the entry point;

drawing the vascular access port towards, until engaging with, the port body extension.

In some embodiments, the step of coupling the vascular access port to the introducer includes:

coupling the port body extension to the introducer,

and/or

fastening to the port body extension an introducer fastener provided at the introducer distal end,

fastening to the vascular access port a shaft fastener provided at a distal end of a shaft extending through the introducer.

In some embodiments, the unified structure is wider than each of the vascular access port and the port body extension before the connecting.

In some embodiments, the step of connecting the vascular access port to the port body extension forces enlarging a portion of the subcutaneous passage adjacent to the unified structure.

In some embodiments, the unified structure has a generally triangular shape defined by side periphery thereof. Optionally, a vertex of unified structure points generally towards the entry point after the connecting.

In some embodiments, a first portion of the port body extension is connectable to a first side of the vascular access port and a second portion of the port body extension is connectable to a second side of the vascular access port.

In some embodiments, the vascular access port includes a port body coupled to a septum member covering a cavity defined by the port body. Optionally, the port body is connectable to or includes an expandable element configured to increase size, area and/or volume of the vascular access port around the septum member when expanded.

In some embodiments, the method further comprising:

introducing a catheter into a vascular system of the subject's body via the first incision.

In some embodiments, a proximal end of the catheter is provided readily affixed to the vascular access port and having a lumen thereof in fluid communication with the cavity.

In some embodiments, the method further comprising:

connecting a proximal end of the catheter to a catheter connector of the vascular access port thereby forming fluid communication between a lumen of the catheter and the cavity.

In some embodiments, the first incision is made superiorly to a collarbone of the patient's body and the entry point is made inferiorly to the collarbone.

According to an aspect of some embodiments of the present invention, there is provided a vascular access port, which comprises:

a port body coupled to a septum member covering a cavity defined by the port body;

a first connector releasably connectable to a shaft fastener, and

a second connector enclosing a connector lumen configured to facilitate fluid communication between the cavity and a lumen of a catheter, when the catheter is connected thereto.

In some embodiments, the port body has a generally triangular shape defined by side periphery thereof, wherein the first connector protrudes from a vertex of port body and the second connector protrudes from a rear side of the port body opposing the port body vertex.

In some embodiments, the second port body connector is readily connected to the catheter.

In some embodiments, the port body is interlockable with a port body extension to form a unified structure having a final shape and size.

In some embodiments, the unified structure is wider than the vascular access port.

In some embodiments, the unified structure has a generally triangular shape defined by side periphery thereof. Optionally, a vertex of unified structure includes a third connector releasably connectable to an introducer fastener.

In some embodiments, a first portion of the port body extension is connectable to a first side of the vascular access port and a second portion of the port body extension is connectable to a second side of the vascular access port.

According to an aspect of some embodiments of the present invention, there is provided a kit which comprises:

the vascular access port;

the catheter; and

a shaft comprising the shaft fastener at a distal end thereof.

In some embodiments, the kit also includes:

a port body extension interlockable with the port body to form a unified structure having a final shape and size; and

an introducer comprising the introducer fastener at a distal end thereof, the introducer includes a tubular body, wherein the shaft is fixatable to the introducer such that the shaft distal end protrudes from within the tubular body.

All technical or/and scientific words, terms, or/and phrases, used herein have the same or similar meaning as commonly understood by one of ordinary skill in the art to which the invention pertains, unless otherwise specifically defined or stated herein. Exemplary embodiments of methods (steps, procedures), apparatuses (devices, systems, components thereof), equipment, and materials, illustratively described herein are exemplary and illustrative only and are not intended to be necessarily limiting. Although methods, apparatuses, equipment, and materials, equivalent or similar to those described herein can be used in practicing or/and testing embodiments of the invention, exemplary methods, apparatuses, equipment, and materials, are illustratively described below. In case of conflict, the patent specification, including definitions, will control.

### BRIEF DESCRIPTION OF THE DRAWINGS

Some embodiments of the present invention are herein described, by way of example only, with reference to the accompanying drawings. With specific reference now to the drawings in detail, it is stressed that the particulars shown are by way of example and for purposes of illustrative description of some embodiments of the present invention. In this regard, the description taken together with the accompanying drawings make apparent to those skilled in the art how some embodiments of the present invention may be practiced.

FIGS. 1A-1B schematically illustrate respectively a side cross-sectional view and a top cross-sectional view of an exemplary deployed vascular access port, in accordance with some embodiments;

FIGS. 2A-2C schematically illustrate respectively side cross-sectional view and top views of an exemplary subcutaneously formable vascular access port, in accordance with some embodiments;

FIGS. 3A-3C schematically illustrate respectively side cross-sectional view and top views of another exemplary subcutaneously formable vascular access port, comprising a first port body member connectable to a laterally expandable second port body member, in accordance with some embodiments;

FIGS. 4A-4H schematically illustrate isometric views of a first exemplary implant comprising a first and a second members with interlocking edges, in accordance with some embodiments;

FIGS. 5A-5C schematically illustrate respectively top views of an exemplary toggling vascular access port in exemplary delivery configuration (FIG. 5A) and alternative exemplary deployment configurations (FIGS. 5B and 5C), in accordance with some embodiments;

FIGS. 6A-6B illustrate respectively an axonometric view, a top exploded view and an axonometric exploded view of another exemplary toggling vascular access port, in accordance with some embodiments;

FIGS. 7A-7B illustrate respectively top views of the toggling vascular access port of FIG. 6A in a delivery configuration and a deployed configuration, in accordance with some embodiments;

FIGS. 8A-8B illustrate respectively top view and axonometric view of an exemplary arm of the toggling vascular access port of FIG. 6A, in accordance with some embodiments;

FIGS. 9A-9C illustrate axonometric views of exemplary inferior portion without (FIG. 9A) and with left arm of the toggling vascular access port of FIG. 6A in a delivery configuration (FIG. 9B) and a deployed configuration (FIG. 9C), in accordance with some embodiments;

FIG. 10 illustrates to view of an exemplary delivery apparatus of the toggling vascular access port of FIG. 6A, in accordance with some embodiments;

FIGS. 11A-11 B illustrate top views of the delivery apparatus of FIG. 10 equipped with the toggling vascular access port of FIG. 6A in a delivery configuration and a deployed configuration, respectively, in accordance with some embodiments;

FIGS. 12A-12C illustrate axonometric views of a portion of the delivery apparatus of FIG. 10 showing grasping mechanism to the toggling vascular access port of FIG. 6A, in accordance with some embodiments;

FIGS. 13A-13C illustrate axonometric views of another exemplary toggling vascular access port comprising a port clamping portion, in accordance with some embodiments;

FIGS. 14A-14B illustrate respectively a top view and an axonometric view of the toggling vascular access port of FIG. 13A clamped with medical forceps, in accordance with some embodiments;

FIG. 15 illustrates an axonometric view of an exemplary toggling vascular access port comprising another exemplary configuration of a port clamping portion, in accordance with some embodiments;

FIGS. 16A-16H schematically illustrate several views representing possible scenarios in execution of a method for subcutaneously delivering and implanting a vascular access port, in accordance with some embodiments;

FIGS. 17A-17C illustrate an exemplary vascular access port before (FIGS. 17A & 17B) and after (FIGS. 17C & 17D) interlocking with an exemplary port body extension, in accordance with some embodiments;

FIGS. 18A-18D illustrate several views representing possible scenarios in execution of a method for deploying the vascular access port shown in FIG. 17C, using an exemplary delivery device, in accordance with some embodiments;

FIGS. 19A-19D schematically illustrate optional members in an exemplary kit for deploying and implanting a vascular access port, in accordance with some embodiments;

FIGS. 20A-20D schematically illustrate several views representing possible scenarios in execution of a method for subcutaneously delivering and implanting a vascular access port using the exemplary kit of FIGS. 19A-19D, in accordance with some embodiments;

FIGS. 21A-21B schematically illustrate two exemplary variations of the exemplary kit of FIGS. 19A-19D for deploying vascular access ports with exemplary anchoring members, in accordance with some embodiments;

FIGS. 22A-22B illustrate respectively a full axonometric view and a partial cut axonometric view of an exemplary tunneling and port delivery apparatus comprising an implantable tunneler tip, in accordance with some embodiments; and

FIGS. 23A-23B illustrate respectively a top view of the tunneling and port delivery apparatus of FIG. 22A equipped with an exemplary vascular access port, and axonometric views of the exemplary vascular access port before and after integration with the implantable tunneler tip thereof, in accordance with some embodiments.

### DETAILED DESCRIPTION

The present disclosure, in some embodiments thereof, relates to devices and methods for facilitating and/or improving repeated deliveries of fluids (e.g., fluids carrying nutrients, medicament and/or agents such as chemotherapy agents) into vasculature of a subject, and more particularly, but not exclusively, to vascular access ports and methods of delivery and deployment thereof in a body of a subject. In some embodiments, vascular access ports of the present disclosure can improve safety and/or efficacy of the surgical implantation procedure of access port and catheter by reducing size or number of surgical processes (like cuts, incisions, and tunneling), their duration and/or complexity, thereby also provide a less traumatic experience and easier recovery for the patient.

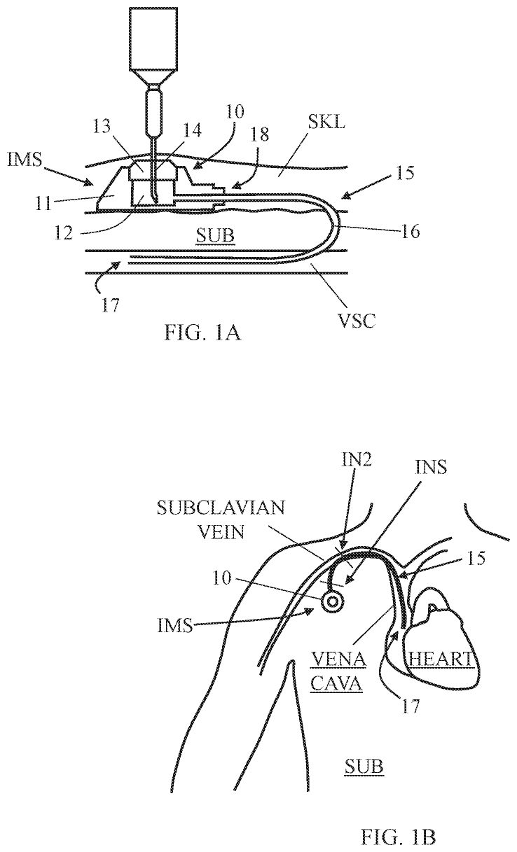

FIGS. 1A-1B schematically illustrate respectively a side cross-sectional view and a top cross-sectional view of an exemplary vascular access port 10, optionally formed as a toggling vascular access port, in a deployed configuration. Port 10 is implanted in a target implantation site IMS subcutaneously beneath skin layers SKL (including optionally within or beneath fat tissue) in a subject SUB. Vascular access port 10 includes a port body 11 defining a cavity 12 and coupled with a septum member 13 that covers and seals cavity 12 from surroundings. Septum member 13 is configured for repeated puncturing of needles, like needle 14 shown in FIG. 1A, without compromising sealing of cavity 12 during needle placement therethrough and after the needle is withdrawn.

“Repeated” in this context may refer to more than 10 consecutive needle punctures, optionally more than 100 consecutive needle punctures, optionally more than 1,000 consecutive needle punctures, optionally more than 10,000 consecutive needle punctures, or higher or lower. “Needle” in this context may refer to needles approved for fluid deliveries through vascular access ports, such as for intravenous administration.

When fully deployed, vascular access port 10 has cavity 12 in fluid communication with vasculature VSC of subject SUB, normally a large blood vessel such as the Subclavian vein or the Vena Cava, so that fluid administrated into cavity 12 via needle 14 will flow directly to the subject's vascular system. A catheter 15 with catheter lumen 16 has a first catheter end 17 thereof positioned in and opened to vasculature VSC, and a second catheter end 18 thereof is connected to port body 11 and opened to cavity 12; catheter ends, 17 and 18, are opened to catheter lumen 16 and facilitate fluid communication between cavity 12 and vasculature VSC. FIG. 1B shows an optional deployment scheme where port 10 is positioned on an upper part the subject's chest in proximity to access opening made to Jugular vein, with first catheter end 17 positioned in the Vena Cava in proximity to subject's right atrium. Vascular access port 10 may be provided separately to catheter 15 with a connector configured for selective connection therebetween, optionally within the body, or alternatively vascular access port 10 and catheter 15 are provided together as an assembly kit or as a unified device.

As used herein, the term “vascular access port” refers to one or more implantable components that together are intended for use after their implantation to repeatedly transfer fluids administered to and/or withdrawn from a subject. The disclosures described herein are advantageous also when used in conjunction with vascular access ports that have a septum member configured for repeated puncturing by a needle, but this particular feature is not a requirement and other forms of needle access openings or platforms may apply. Some vascular access ports described herein include one or more components configured, collectively, when properly assembled and deployed, for prolonged implantation in a live (e.g., human) subject and for repeated fluid transfer access, such as through a septum member. The vascular access port includes at least a structural object referred to herein as a “port body” which serves as a facilitating structure for fluid transfer access and/or as a support structure configured for holding components (e.g., a septum) applicable for fluid transfer access. In some embodiments, the port body forms a cavity beneath (e.g., inferiorly to) the needle access opening or septum, which is sized and shaped for repeatedly receiving a needle tip, for accumulating a chosen or predetermined volume of fluid (e.g., a liquid such as a solution, a suspension or a colloid), and/or for fluid administration to, and/or withdrawal from, a vasculature of the live subject. In some embodiments, a vascular access port may include a single cavity or several distinct cavities, covered with one or several distinct septum members, provided as a single element or as several interconnectable members, some or all can be provided in the port body or in several portions or members of the vascular access port configured each as a separate port body. Before or after implantation, a catheter may be attached to the vascular access port with a distal end that physically enters the vasculature of the patient. Once connected, a lumen of the catheter is provided in direct fluid communication with the port body cavity. A “vascular access port” as described herein, or a kit comprising it, may include or not include such a catheter and may include or not include a fitting for such a catheter. A vascular access port may be referred to herein as simply a “port” or an “implant”. A vascular access port may have additional components and functionality not associated with fluid delivery or withdrawal.

A “toggling vascular access port” refers to any type of vascular access port comprising at least one object (e.g., a first portion, member, or extension of the vascular access port), movably connected or connectable to another object (e.g., a second portion, member, or extension of the vascular access port), in a way that restricts relative motion therebetween to a combination of rotation and translation of one relative to the other. A toggling vascular access port, for example, may have the at least one object restricted to sliding motion over a surface of the other object while also rotating around a rotation axis. The rotation axis may be located within the toggling vascular access port or remote thereto, including for example beyond the surface of the other object.

The port body may be structurally and/or functionally configured for facilitating at least the basic function of the vascular access port of repeated accumulating and delivering and/or withdrawing fluid to or from subject's vasculature, and it may optionally lack or be initially configured without one or more other features, optional or vital ones, for facilitating additional functions associated with delivery, deployment and/or prolonged use of a vascular access port. The port body may be connected to at least one other component for providing the vascular access port additional features or capabilities, for example improved or easier deliverability, selective fixation to body tissues surrounding the port body and/or increased stability in a chosen implantation site such as a preformed subcutaneous void.

In some embodiments, the vascular access port, or the port body itself, must be changed or reconfigured (such as by way of reshaping or expanding portions thereof, by assembling from sub-components thereof, or by shifting components or sub-components thereof or connected thereto) before or during deployment of the vascular access port. Such changes of the port body and/or of the vascular access port may be accomplished within the body of the subject, optionally in a subcutaneous location in the body (e.g., beneath one or more layers of the subject's skin).

The term “port body” may refer to one or more components out of a plurality of components forming the vascular access port, or it may refer to the collection of these components that together are configured to form a the vascular access port. The term “port body” may also refer to only the component of a complete port body that contains the part of a vascular access port configured for fluid transfer access and/or delivery and withdrawal. For example, a “port body” may be referred to as only the septum containing or holding component, the cavity forming component, and/or the catheter connecting component, of a complete port body for a vascular access port.

When deployed to form a vascular access port suitable for use in fluid delivery or withdrawal, the coupled/engaged collection of port body components, including the “port body” or components thereof, may be referred to herein as a “unified structure” or “deployed configuration” of the vascular access port. Prior to deployment, these components may be fully engaged, fully disengaged, or partially disengaged, and this may include for example being unconnected with each other, easily movable one relative to the other, and/or separated from each other with or without a gap therebetween. The coupling or engagement functionality may be provided by all or some or only one of the separable port body components. The components of a port body may be referred to herein generically such as the first port body member and second port body member, etc. In some embodiments, components of the vascular access port, other than the port body, are designated with other functional associations such as “port body extension.” Of course, descriptive functional or structural designations could be applied to each part of a multi-component port body such as “second member”, “septum support,” “expander,” “retainer,” “base,” or the like.

Deploying the vascular access includes at least inserting the port body into a target implantation site in the subject body, such that a posterior portion of the port body is accessible to repeated fluid transfer access. Vascular access port deployment then includes compacting of tissue mass surrounding periphery of the port body thereby increasing a volume of a void formed in the target implantation site between the periphery of the port body and the compacted tissue mass. The void can be a subcutaneous void located between or beneath skin tissue layers at the target implantation site. Concurrently with increasing the void volume, or immediately afterwards, the increased void volume is occupied with the vascular access port such as by increasing the volume of the port body or by connecting one or more solid shaped components (e.g., a port body extension) thereto. This also includes the situation that the tissue mass compaction may be a direct result of such increase in port body volume. The compacted tissue mass normally affects a continuous pressure on the deployed vascular access port and thereby increase its fixation and/or stability in the subcutaneous void. The port body may include an inferior portion which defines a cavity and a posterior portion coupled with a septum member covering the cavity, and the vascular access port may be deployed such that the compacted tissue mass surrounds only an inferior portion and not the posterior portion of the port body.

The vascular access port is optionally inserted to the target implantation site (e.g., into the void) when provided in a delivery configuration, wherein a port body extension thereof is partially or wholly disengaged from the port body, and occupying the increased void volume is accomplished by changing (e.g., transferring) the vascular access port into a deployed configuration wherein the port body and the port body extension are forced to adjoin and fixedly connect to form a unified structure of the vascular access port being greater in volume than the port body. Optionally and alternatively, a portion of the port body can be expanded to occupy (and optionally form) the increased void volume.

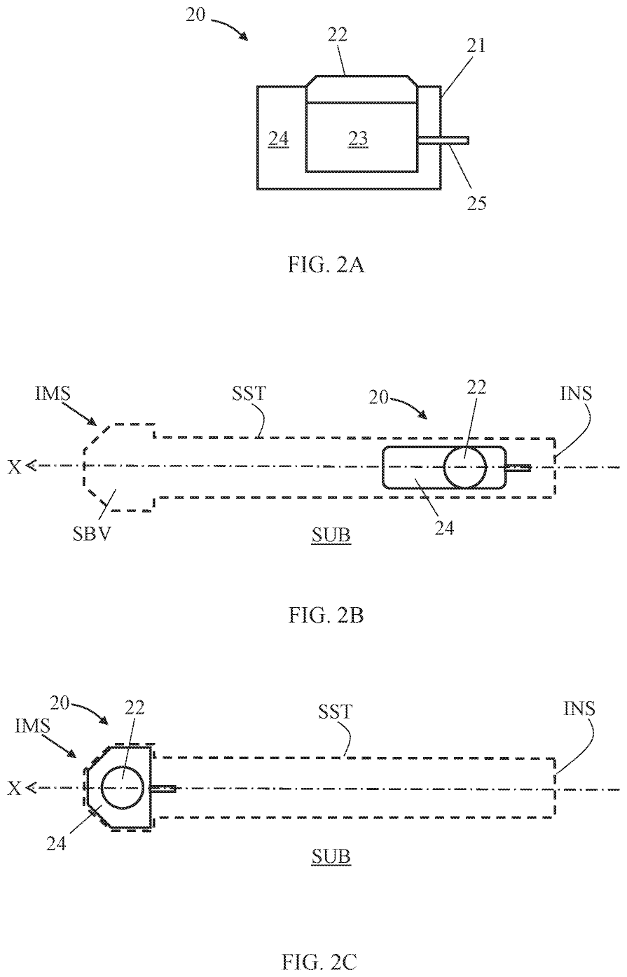

Reference is made to FIGS. 2A-2C which schematically illustrate respectively side cross-sectional view and top views of an exemplary subcutaneously formable vascular access port 20. Port 20 may be configured similar, identical or equivalent in function, structure and/or dimensions to port 10 and/or configured to be placed into the proximal portion of the incision similarly thereto, with or without catheter 15.

Port 20 includes a port body 21 coupled with a septum member 22 covering a cavity 23 defined by port body 21. Port body 21 is provided in a delivery configuration (shown in FIGS. 2A and 2B), having an elongated form extending along a longitudinal axis X, and is selectively changeable to a deployed configuration (as shown in FIG. 2C) by changing in size parallel to the longitudinal axis X. Change in size may be achieved by decreasing in length along longitudinal axis X, optionally from a first predetermined length to a second, smaller, predetermined length. Port body 21 is further configured to change in size transversely to the longitudinal axis X, when changing from the delivery configuration to the deployed configuration.

Port body 21 is connected to, connectable to, or includes, an expandable element or portion, referred to herein as a port body extension 24, that is configured to increase size, area and/or volume of vascular access port 20 around septum member 22 when expanded and/or moved relative to port body 21, as shown in FIG. 2C. Port body extension 24 may include or be configured as an inflatable member, a mesh, a preloaded elastic member, or a bellows shaped structure. Optionally, alternatively or additionally, port body 21 is connected to an at least one port body extension 24 that can move relative thereto, having a combined form changeable between a longer and narrower form (e.g., a delivery configuration) to a shorter and wider united (e.g., a deployed configuration). Vascular access port 20 may expand or increase in cross section or volume by way of self-expansion or deformation, or by connecting with one or more appendix members connectable thereto to form a unified structure being greater in length, width and/or height than the port body alone in the delivery configuration. In some such embodiments, port body 21 is connectable and/or selectively movable relative to a port body extension 24 (as will be described with respect to vascular access port 100 and toggling vascular access port 300, for example) to form a unified structure having a final shape and size.

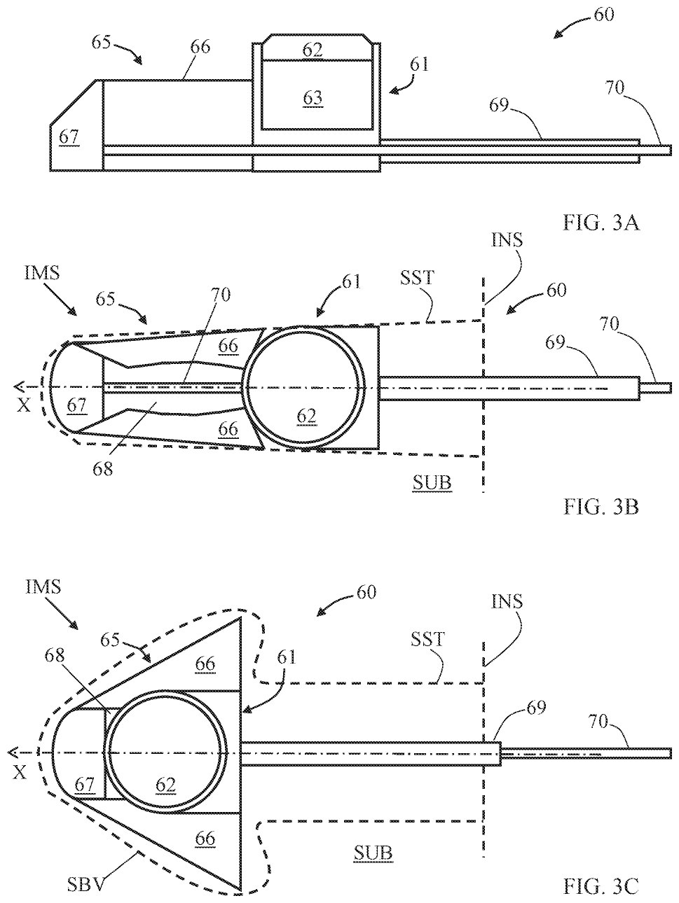

The port body 21 either before or after deployment may be configured in an essentially unlimited variety of flat or curved solid shapes such as prisms, pyramids, cubes, spheres, ellipsoids, ovoids, and the like, in any combination thereof, either alone or as augmentations to other shapes. In some embodiments, the final shape of vascular access port 20, if as a unified structure or in its basic structure, is generally triangular as defined by side periphery thereof. The phrase “generally triangular,” as used herein, encompasses any generally three-sided geometry wherein adjacent sides intersect, including but not limited to regular or irregular polyhedron and/or curved solid with one or more three edge bound faces, The term “generally triangular” may include shapes with rounded edges, curved faces, and other deviations from linear and planar geometric components, for example three-sided polygons, circular triangles and equilateral triangles. For example, the wedge shapes shown in FIGS. 3C, 4E and 7B discussed below are examples of generally triangular shaped ports. Port body 21 may include a single cavity or several distinct cavities, covered with one or several distinct septum members, provided as a single element or as several interconnectable members.

For deploying vascular access port 20 in a body of a subject (e.g., subject SUB), an incision INS is made in proximity to a target implantation site (e.g., implantation site IMS) in the subject body. A subcutaneous void SBV (e.g., in a form of pocket) is formed between or beneath skin tissue layers (e.g., skin tissue layers SKL) at target implantation site IMS. Vascular access port 20 is inserted into the subcutaneous void SBV when port body 21 is provided in the delivery configuration (FIG. 2B). Vascular access port 20 is then deployed by changing port body 21 into the deployed configuration by decreasing its size along longitudinal axis X (FIG. 2C). The subcutaneous void SBV may be initially formed, or increased in size, as a result of inserting port 20 by forcing port body 21 within the subject body through incision INS. Increasing size of subcutaneous void SBV can result in compaction of tissue mass surrounding port body 21, thereby increasing fixation and/or stability of port 20 in the target implantation site IMS.

As part of port 20 deployment, or as a distinct step, catheter 15 is provided and implanted for providing fluid communication between cavity 23 and vasculature VSC of subject SUB (similarly to as shown in FIGS. 1A and 1B and as described above).

Optionally, a subcutaneous surgical tunnel SST is created between incision INS and target implantation site IMS, and port 20 is delivered through the surgical tunnel SST to the subcutaneous void SBV, and optionally no other incision is made for port (and catheter) deployment. Optionally and alternatively, incision INS is located at the surface of, and serves as opening directly into, implantation site IMS, without having a surgical tunnel extending and connecting therebetween. In some such embodiments, first catheter end 17 is inserted into the subject body via a second incision IN2 remote to incision INS (as shown in FIG. 1B), and subcutaneous surgical tunnel SST is created between second incision IN2 and incision INS. Optionally, second catheter end 18 is delivered through the surgical tunnel SST towards from second incision IN2 to incision INS if for connecting with port 20 already present in subcutaneous void SBV (e.g., by coupling with a connector 25), or already connected with port 20 and delivered together therewith to implantation site IMS.

The embodiment shown in FIGS. 2A-2C illustrates some general principles of many advantageous embodiments of vascular access ports described herein. One advantageous feature is that the ratio of the externally exposed surface area to the total enclosed volume of the originally delivered components of the vascular access port may be lower in the deployed configuration than in the delivery configuration. This change in geometry is very different from vascular access port stabilization methods that use spikes or plates or other projections that are made to extend outward from the port body portion containing the septum. While these may be somewhat beneficial, improvement in port stability is much greater when the total unified structure changes geometry to decrease surface area to volume ratio rather than increase it as spikes or plates will do.

A reduction in surface area to volume ratio upon deployment can be accomplished in a variety of ways. In some embodiments, the shape can stay substantially the same when deployed but the dimensions can increase, such as described above where an expandable element 24 may be provided around the port body portion with the septum. Another approach can be to change the shape of the vascular access port to distribute the volume more evenly in all three spatial dimensions. This occurs, for example, in the shape transition from FIG. 2B to 2C discussed above. In other embodiments, the reduction in surface area to volume ratio may be accomplished by engaging upon deployment substantial segments of the surface areas of two or more components of the vascular access port that are delivered in a disengaged or only partially engaged state. This reduces the externally exposed surface area of the unified structure without reducing the sum total enclosed volume of the components. This further enhances the fixation and stability of the vascular access port within the implantation site IMS.

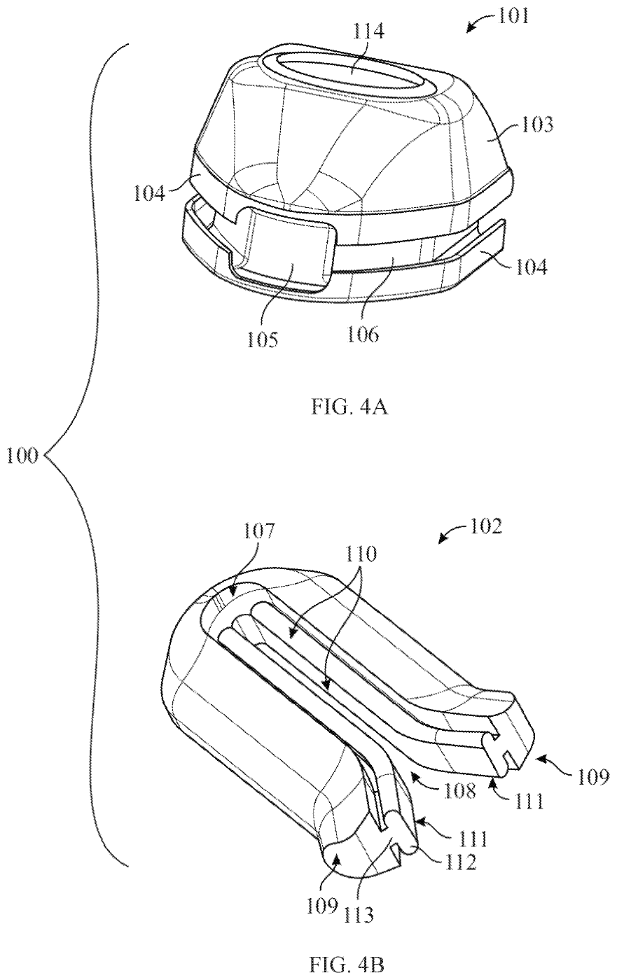

Independent of any changes in surface area to volume ratio between delivery and deployed configurations, in some embodiments the volume of the structure after deployment is significantly larger than the volume of any of the one or more delivered components prior to deployment. As discussed above, this allows the deployed configuration to become solidly affixed in the implantation site IMS. One way of accomplishing this is utilizing an expandable portion such as described in one exemplary embodiments of the port body extension 24 of FIG. 2. Also, as shown and discussed below with respect at least to FIGS. 3 and 4, this volume increase may be accomplished by engaging two components of a port body that are wholly or partially disengaged in the delivery configuration. Another aspect of the change from delivery to deployed configuration may be an increase in base area of the deployed vascular access port over the delivery configuration. This also improves fixation of the port after deployment. All three of these delivery to deployment changes, surface area to volume ratio reduction, volume increase, and base area increase, can be provided independently or in any combination of two or more in any given embodiment.

Another advantageous feature of some embodiments described herein is that the transition from delivery configuration to deployed configuration may be accomplished with a linear sliding motion along the implantation incision to the implantation site/void. Optionally, only the linear sliding motion is required which may involve a combination of rotation and translation of an at least one port body extension relative to the port body. No threads requiring a rotating motion, no fasteners, no separate catheter or tool access procedures are required in order to engage the components of the vascular access port in accordance with the principles described herein.

Furthermore, in some embodiments, the port body extension is implanted first. This component may be a rigid, semi-rigid or flexible polymer (e.g., silicone or a monolithic plastic, for example) or metal part with no fluid delivery functionality. This component can be easier to install without having to evaluate orientation issues relating to functionality. Once the port body extension is implanted or positioned in the target implantation site, the port body with the septum can be inserted to the target implantation site to engage the already correctly implanted extension.

FIGS. 3A-3C schematically illustrate respectively a side cross-sectional view and top views of a subcutaneously formable vascular access port 60. Port 60 may be a variation of port 20 and may be configured similar, identical or equivalent in function, structure and/or dimensions to either one of ports 10 and 20, and/or deployed similarly thereto, with or without catheter 15. Port 60 includes a port body 61 that is coupled with a septum member 62 covering a cavity 63 entirely defined by port body 61. Port body 61 may also be referred to as a septum support 61. Cavity 63 has an inlet at its upper (posterior) portion thereof which is closed with septum member 62, and an outlet (fluidly connected to the inlet) in a lower (inferior) portion thereof in proximity to rear (proximal) end of port body 61 which is optionally opened to a connector (similar to connector 25 shown in FIG. 2A, for example) configured for providing fluid communication between the cavity and a lumen of a catheter or any other fluid delivery medium. Vascular access port 60 is provided in a delivery configuration (shown in FIGS. 3A and 3B), having an elongated form extending along longitudinal axis X, and is selectively changeable to a deployed configuration (as shown in FIG. 3C) by changing in size parallel to longitudinal axis X.

Port 60 further includes a second port body member configured as a port body extension 65 connectable with port body 61 and the two members (port body 61 and port body extension 65) are provided one after the other along longitudinal axis X, connected or disconnected, and may be at least partially separated from each other when vascular access port 60 is in the delivery configuration. Port 60 is selectively changeable subcutaneously from the delivery configuration to a deployed configuration wherein port body extension 65 adjoins and is fixedly connected to port body 61 to form a unified structure of vascular access port 60, being greater in volume, width and/or length than port body 61, optionally by at least 10%, or by at least 15%, or by at least 30%, or by at least 50%, in volume. Port body extension 65 is configured to stabilize and/or fixate port body 61, when port 60 is in the deployed configuration, by forming a unified structure shaped and sized effectively for increasing support and stability of port 60, relative to shape and size of port body 61 before fixedly connecting with port body extension 65, when implanted in a subcutaneous void in a subject's body.

Although in some embodiments port body 61 is functionable as a vascular access port, it may lack sufficient stability due to size and/or shape thereof within the subcutaneous void, and this way can be more prone to migrate or turn over if not properly fastened to the body. Furthermore, port body extension 65 is configured to expand when assembling with port body 21, such that the unified structure of port 60 as a whole enlarges within the subcutaneous void stretching it further or even causing further dissection of tissues in a manner that increases stability and/or fixation within the subcutaneous void, optionally without need for applying fastening or suturing of port 60 to surrounding tissue.

Port body 61 is configured to affect change in shape and/or size of port body extension 65 by engaging, joining and fixedly connecting to port body extension 65, when port 60 changes to the deployed configuration. As part of the changing process of vascular access port 60 from the delivery configuration to the deployed configuration, port body 61 is moved distally, optionally by force applied by the operator, towards port body extension 65, optionally diminishing a gap formed between respective boundaries of port body 61 and port body extension 65 in the delivery configuration. When properly joined and pressed against each other, the two members fixedly connect by having port body 61 interlocking with port body extension 65.

Port body 61 is configured to deform port body extension 65 and force it to expand laterally, when vascular access port 60 changes to the deployed configuration. Port body extension 65 includes two flexible portions 66 extending backwardly (towards port body 61, in the delivery configuration) from a front edge 67. Port body extension 65, with flexible portions 66 thereof, enclose an inner surface shaped in accordance with outer surface of port body 61 such that, when port body 61 is pushed or pressed against the inner surface of port body extension 65, flexible portions 66 rotate outwardly (away from longitudinal axis X, respectively) about front edge 67, causing port body extension 65 to laterally expand, and eventually embrace outer surface of port body 61. Optionally, port body extension 65 is deformable under force applicable by port body 61 thereto when port body 61 is pushed against its inner surface, such that the inner surface conforms at least partially to a shape imposed by the outer surface of port body 61, whereas port body 61 is configured to maintain size and shape thereof when pressed against port body extension 65.The final shape of vascular access port 60 in its unified structure resembles a generally triangular shape (in top view), as shown in FIG. 3C, optionally a three sided polygon, a circular triangle, or an equilateral triangle, while port body 61 may resemble a non-triangular shape, as shown in FIG. 3B. Since that flexible portions 66 of port body extension 65 undergo rotation and translation relative to port body 61, when vascular access port 60 changes from the delivery configuration to the deployed configuration, until fixedly connecting thereto to form a unified structure, vascular access port 60 is considered a toggling vascular access port as defined above.

Front edge 67 is optionally configured for surgically forming a subcutaneous tunnel beneath skin layer of a live subject, and/or for forming a subcutaneous void for implanting vascular access port 60 therein. Edge 67 is optionally rounded or chamfered, pointing away from port body 61, relative to the longitudinal axis X, and is configured for affecting atraumatic tissue layers separation when forced to pass therebetween. Each of flexible portions 66 may also have a rounded or chamfered edge pointing laterally outwardly relative to longitudinal axis X, such that upon outward rotation they may affect stretching and/or further atraumatic tissue layers separation when forced against tissue layers forming walls of subcutaneous void housing port body extension 65, during change of vascular access port 60 to the deployed configuration. Relative (axial) motion between port body 61 and port body extension 65, and interlocking by pressing them against each other, is accomplished using shafts which can be manipulated at a remote proximal location depending on their length, optionally including a first shaft 69 that is connected with distal end thereof to port body 61, and a second shaft 70 that is connected with a distal end thereof to port body extension 65.

For changing into the deployed configuration, port body 61 moves towards port body extension 65 along longitudinal axis X thereby optionally diminishing a gap 68, formed between respective boundaries of port body 61 and port body extension 65 and with respective flexible portions 66. Gap 68 may be at least partly covered and/or filled with a compressible substance, a fluid, a flexible material or a viscoelastic material. Relative motions between port body 61 and port body extension 65 may be restricted to a predefined path relative longitudinal axis X.

Port 60 can be configured for allowing or restricting to one or more operational maneuvers of shaft(s) 69 and/or 70, for example: facilitating/allowing manipulation of second shaft 70 to advance port body extension 65 until it reaches or exceeds the target implantation site (and optionally surgically forms or increases volume of the subcutaneous void), then optionally fixating second shaft 70 and/or port body extension 65 in place, then facilitating/allowing manipulation of first shaft 69 to advance port body 61 until it reaches and joins port body extension 65. Optionally and alternatively, vascular access port 60 is configured to allow more flexibility and the operator can optionally implement one of different schemes to manipulate shafts 69 and 70, separately and/or together.

For deploying vascular access port 60 in a body of a subject (e.g., subject SUB), an incision INS is made in proximity to a target implantation site (e.g., implantation site IMS) in the subject body. Optionally, a subcutaneous surgical tunnel SST is created between incision INS and target implantation site IMS, and port 60 is delivered through the surgical tunnel SST to the subcutaneous void SBV. The surgical tunnel length is optionally a few centimeters in length, optionally greater than 5 cm, for example. Optionally, port 60 and parts thereof (including port body 61 and port body extension 65) are delivered through a lumen of an elongated delivery device, optionally in a form of a surgical tunneler, extending along surgical tunnel SST. The elongated delivery device may include a rail and may be configured to restrict motion of port body 61 relative to port body extension 65 in a chosen direction therealong, such as to travelling along the rail.

A subcutaneous void SBV is formed between or beneath skin tissue layers (e.g., skin tissue layers SKL) at target implantation site IMS optionally using a dedicated device, the tunneler or the front end of port 60, for example. Vascular access port 60 is inserted into the subcutaneous void SBV when port body 61 is provided in the delivery configuration (FIG. 3B). First body extension 65 is first to enter subcutaneous void SBV, which is already present or formed with front edge 67. Delivery is performed when vascular access port 60 is in delivery configuration, so port body 61 and port body extension 65 are passed either together (while maintaining gap 68, or a chosen distance, therebetween) or one after the other, to the target implantation site IMS. Vascular access port 60 is then put into the deployed configuration. The subcutaneous void SBV is forced into a greater volume as a result of assembling port 60 and lateral expansion of port body extension 65 within subcutaneous void SBV. Increasing the volume of subcutaneous void SBV can result in compaction of tissue mass surrounding port body extension 65, thereby increasing fixation and/or stability of vascular access port 60 in the target implantation site IMS.

As part of port 60 deployment, or as a distinct step, catheter 15 is provided and implanted for providing fluid communication between cavity 63 and vasculature VSC of subject SUB (similarly to as shown in FIGS. 1A and 1B and as described above).

In some other embodiments, incision INS is located at the surface of, and serves as opening directly into, implantation site IMS, without having a surgical tunnel extending and connecting therebetween. In some such embodiments, first (distal) catheter end 17 is inserted into the subject body via a second incision IN2 remote to incision INS (as shown in FIG. 1 B), and subcutaneous surgical tunnel SST is created between second incision IN2 and incision INS. Optionally, second (proximal) catheter end 18 is delivered through the surgical tunnel SST towards from second incision IN2 to incision INS if for connecting with port 60 already present in subcutaneous void SBV, or already connected with port 60, optionally particularly to first port body member 64, and delivered together therewith to implantation site IMS.

FIGS. 4A-4H schematically illustrate views of an implant 100, and components thereof. Implant 100 may be configured similar, identical or equivalent in function, structure and/or dimensions to either one of vascular access port 10, 20 and 60, and/or deployed similarly thereto, with or without catheter 15. Implant 100 includes a first member 101 (FIGS. 4A and 4C) configured as a port body (similar to port body 61) and a second member 102 (FIG. 4B) configured as a port body extension (similar to port body extension 65). First member 101 and second member 102 are interconnectable with each other (as shown in FIG. 4E) and, when implant 100 is in a delivery configuration, they are optionally provided disconnected and at least partially separated with each other (as shown in FIG. 4D). First member (port body) 101 is functionally configured as a vascular access port and entirely defines a cavity 115 closed with a septum member 114, however connection thereof with second member (port body extension) 102 into a unified structure, as taught hereinafter, is suggested for providing a vascular access port in finalized (deployed) configuration that is advantageous in terms of stability and fixation to implantation site, relative to first member 101 alone. Second member 102 is constructed similarly to port body extension 65 with portions similar to flexible portions 65, which are configured to undergo rotation and translation relative to first member 101, when vascular access port 100 changes from the delivery configuration to the deployed configuration, until fixedly connecting thereto to form a unified structure. Therefore, similarly, vascular access port 100 is considered a toggling vascular access port as defined above.

FIG. 4C shows a cross-sectional view of first member (port body) 101. First member 101 has a posterior portion 103 and an inferior portion 116 enclosed with an external surface. Posterior portion 103 caps cavity 115 and includes an opening to the cavity that is blocked and sealed with septum member 114 connected to posterior portion 103. Inferior portion 116 includes front end 105, a rear end 118, and the base of the port body, and encloses most or all volume of cavity 115. Second member (port body extension) 102 includes an inner surface configured to engage and/or cover front end 105 and sides of inferior portion 116, when implant 100 (vascular access port) is in the deployed configuration. Port body inferior portion 116 comprises two opposing first member edges 104, one on each side thereof, with first member front end 105 located therebetween. Each one of the first member edges 104 has a longitudinally extending groove 106 made laterally inwardly towards centerline of first member 101. Second member 102 encloses an internal surface 107 that is accessible through a rear opening 108 provided on a second member rear 109. Internal surface 107 of second member 102 includes two opposing second member inner edges 110 each having a longitudinally extending ridge 111 projecting laterally therefrom towards centerline of second member 102.

First member 101 is configured to interlock longitudinally within second member 102 when pushed with front end 105 thereof through rear opening 108 and engaging external surface of the first member 101 with the internal surface 107 of second member 102 such that each one of ridges 111 interengages longitudinally with respective groove 106. Front end 105 is optionally equipped with an aligning member 119 projecting axially distally and configured to engage a mating recess in second member 102 thereby fixedly connecting, centering and/or aligning long axes of first and second members 101 and 102. By pushing first member 101 with front end 105 through the rear opening 108 and engaging external surface of first member 101 with internal surface 107, the internal surface 107 is forced to expand until it corresponds in shape and/or size to external surface of first member 101, and/or the second member 102 is forced to expand until the first member 101 interlocks with second member 102.

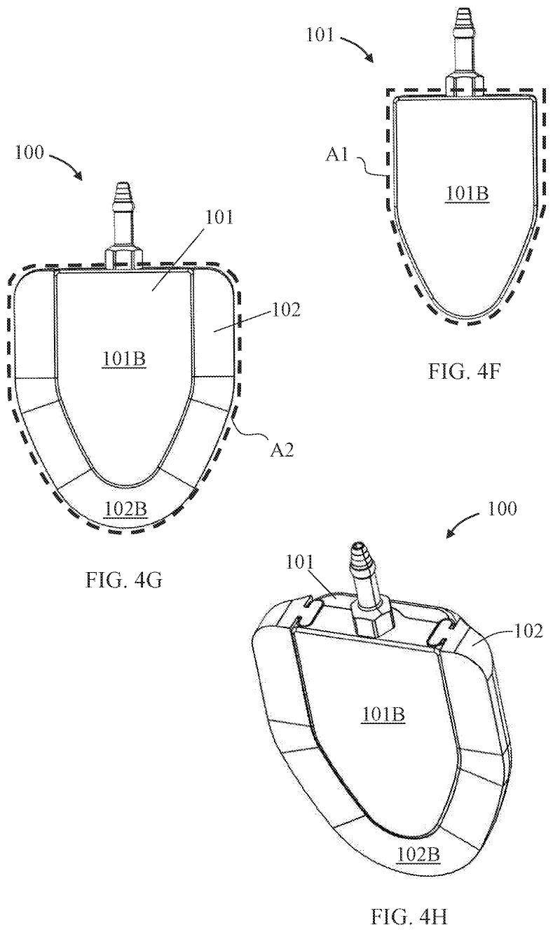

Each one of grooves 106 is aligned, and corresponding in cross-section, to the corresponding ridge 111; optionally but not necessarily all grooves and ridges are aligned. The cross-section shared by each corresponding pair of groove 106 and ridge 111 comprises a wider head section 112 and a narrow neck section 113. When the ridges 111 interengage longitudinally with grooves 106, head section 112 of each ridge cross section nests within the head section 112, and held in place by the neck section 113, of the respective groove cross section. As shown in FIG. 7D, implant 100 is in a fully assembled form when second member 102 surrounds front 105 and first member edges 104 and conforms to shape of external surface of first member 101, and particularly of port body inferior portion 116. In this configuration implant 100 has a final size and shape and is ready for deployment and use.

When in the deployed configuration, rear end 118 of first member 101 (particularly of port body inferior portion 116) is not covered with second member 102 (port body extension). The port body rear end 118 is connected or connectable via a connector 117 to a proximal end of a catheter for facilitating fluid communication between cavity 115 and a lumen of the catheter.

FIGS. 4F-4H show different views showing base (e.g., bottom or inferior) surfaces of first member (port body) 101 before and after assembling with second member (port body extension) 102 to form a unified structure of implant (vascular access port) 100. First member 101 has a first base surface 101B and second member 102 has a second base surface 102B. As shown in FIG. 4F, first base surface 101 B has a first base area A1, and as shown in FIG. 4G, the unified implant 100 has a second base area A2 which is substantially greater than first base area A1. FIG. 4H shows that first base surface 101A and second base surface 102B are substantially flat and/or coincide with each other on same plane such that the unified structure of implant 100 has a substantially flat, level and/or even surface.

In some embodiments, second base area A2 is greater than first base area by at least 15%, optionally by at least 30%, optionally by at least 50%, optionally by at least 75%. In some such embodiments, first base area A1 is smaller than about 500 mm², optionally smaller than about 400 mm², optionally smaller than about 300 mm², optionally smaller than about 200 mm². Optionally, additionally or alternatively, second base area A2 is greater than about 200 mm², optionally greater than about 300 mm², optionally greater than about 400 mm², optionally greater than about 500 mm². In an exemplary embodiment, first base area A1 is about 245 mm² and second base area A2 is about 465 mm², therefore second base area A2 is greater than first base area A1 by about 90%.

The total outer surface area of first member (port body) 101 encloses a first solid shape (shown in FIG. 4A, for example), and the total outer surface area of the unified implant 100, comprising the fully assembled first and second members 101 and 102, encloses a second solid shape (shown in FIG. 4E, for example). In some embodiments, the volume of the second solid shape (e.g., the volume of implant 100 in its unified structure) is greater than the volume of the first solid shape (e.g., the volume of first member 101 only) by at least 15%, optionally by at least 30%, optionally by at least 50%, optionally by at least 75%. In some such embodiments, the volume of the first solid shape is smaller than about 5,000 mm³, optionally smaller than about 3,000 mm³, optionally smaller than about 2,000 mm³, optionally smaller than about 1,500 mm³. Optionally, additionally or alternatively, the volume of the second solid shape is greater than about 2,000 mm³, optionally greater than about 2,500 mm³, optionally greater than about 3,000 mm³. In a first exemplary embodiment, the volume of the first solid shape is about 1,715 mm³ and the volume of the second solid shape is about 2,715 mm³, therefore the volume of the second solid shape is greater than the volume of the first solid shape by about 58%. In a second exemplary embodiment, the volume of the first solid shape is about 1,835 mm³ and the volume of the second solid shape is about 2,700 mm³, therefore the volume of the second solid shape is greater than the volume of the first solid shape by about 47%. Furthermore, the second solid shape (of the unified structure) has a lower surface area to volume ratio than the combined first member 101 (port body) and second member 102 (port body extension) when partially or wholly disengaged in the delivery configuration (as shown in FIG. 4D, for example).

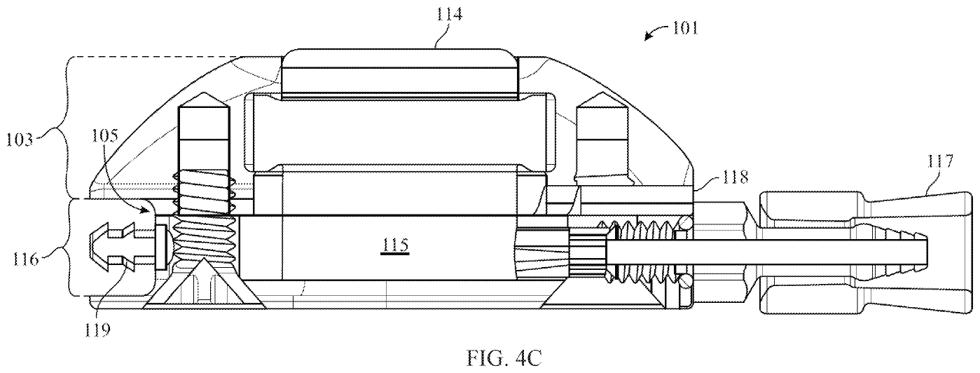

FIGS. 5A-5C schematically illustrate respectively top views of an exemplary toggling vascular access port 200 provided in a surgically premade subcutaneous void or passage SBV in a body of a subject SUB, surrounding target implantation site IMS. Port 200 may be configured similar, identical or equivalent in function, structure and/or dimensions to either one of vascular access port 10, 20, 60 and/or implant 100, and/or deployed similarly thereto, with or without catheter 15. Port 200 is configured to change selectively from a delivery configuration (as shown in FIG. 5A, for example) to a deployed configuration (as shown in FIG. 5B or FIG. 5C, for example) for anchoring to surrounding tissues in target implantation site IMS. Vascular access port 200 includes a port body 201 that is coupled with a septum member 203 covering a cavity defined by the port body. Port 200 also includes an at least one port body extension 202 which can be provided as a single element or connected elements or unconnected elements. Port body extension 202 includes a wide head portion 206 and an at least one body portion, such as narrower laterally opposing body portions 204 and 205, as shown, which extend generally axially from head portion 206 and connect distinctly to port body 201 at laterally opposing portions thereof.

FIG. 5A shows vascular access port 200 in a delivery configuration, in which head portion 206 is initially provided distally to port body 201 with a gap therebetween, and is configured (e.g., in shape and construction) to push, compress or dissect soft tissues when forced thereinto, such as for forming or enlarging subcutaneous void or passage SBV. Furthermore, body portions 204 and 205 are initially provided extending axially and/or are minimally or not protruding laterally with boundaries thereof relative to lateral boundaries of port body 201 or port body extension 202. As such, the overall width of vascular access port 200 remains substantially constant between port body 201 and port body extension 202, optionally about the size of maximal width of port body 201 or of port body extension 202. Body portions 204 and 205 are configured to extend laterally when port body 201 and head portion 206 are brought closer together axially, and they can either plastically or elastically deform to buckle outwardly, can have one or more hinges such that segments thereof can pivot with each other, or are designed in a certain shape and/or construction such that they are forced to separate and/or shift laterally outwardly relative to head portion 206 when forced to travel along lateral boundaries of port body 201. Either way, port body extension 202 and portions 204 and 205 thereof in particular, are configured, once extending laterally outwardly, when in a deployed configuration, to push, compress and/or dissect soft tissues surrounding port 200 when forced to protrude laterally, thereby increasing locally width of subcutaneous void SBV and anchoring port 200 thereto.

As such, port body 201, port body extension 202, integrate therebetween a toggle anchoring mechanism that causes combined rotation and translation motion of body portions 204 and 205, which can be selectively applied by a user to deform port body extension 202 by approximating (i.e., moving closer together) port body extension 202 and port body 201 along a median plane MP of port body 201, and parting the laterally opposing portions 204 and 205 of port body extension 202 transversely to median plane MP, thereby reducing length-to-width ratio of the vascular access port 200. FIG. 5B shows a first example where target implantation site IMS is located in a distal portion of subcutaneous void SBV, and the approximation occurs by pushing port body 201 distally in subcutaneous void SBV while port body extension 202 remains in place (such as by forcing it in place and/or by resistance from surrounding tissue). FIG. 5C shows a second example where target implantation site IMS is in a proximal portion of subcutaneous void SBV, and the approximation occurs by pulling port body extension 202 distally in subcutaneous void SBV while port body 201 remains in place.

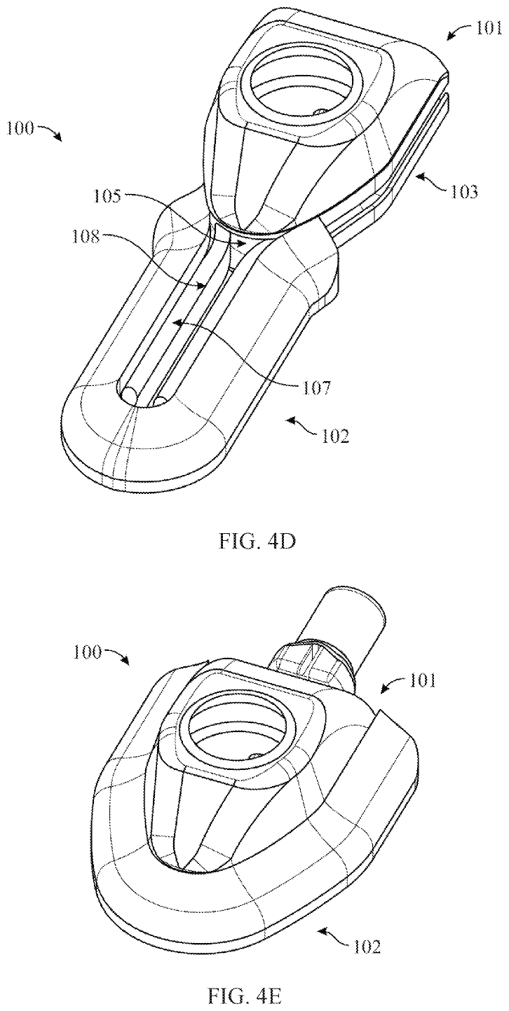

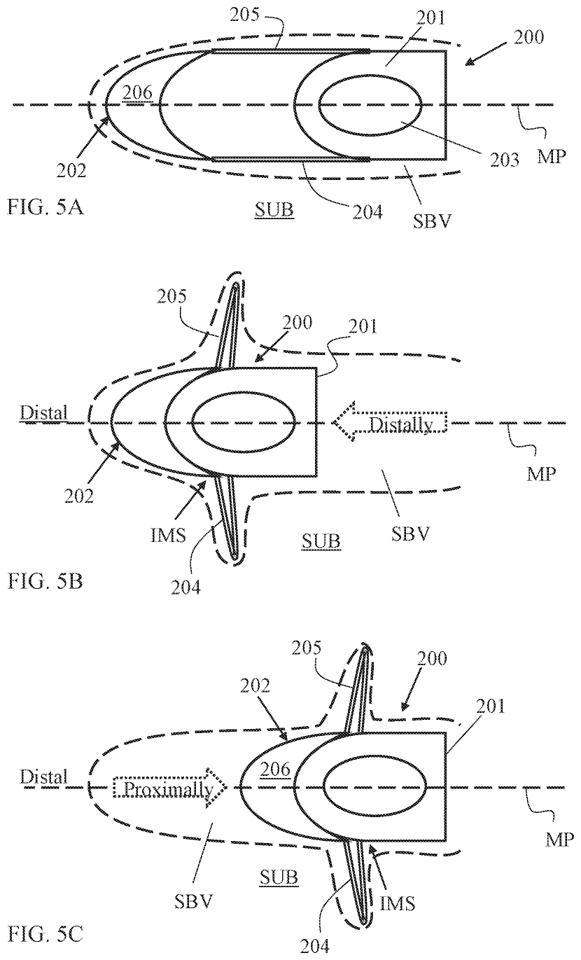

Reference is now made to FIG. 6A that illustrates an axonometric view of a toggling vascular access port 300 in a deployed configuration. Vascular access port 300 is similar in function and/or structure to port 200 and includes a port body 301, port body extensions 304 and a toggle anchoring mechanism 305 configured for selectively changing the vascular access port 300 from a delivery configuration (shown in FIG. 7A, for example) to a deployed configuration (shown in FIG. 7B, for example). FIGS. 6B and 6C illustrate respectively a top exploded view and an axonometric exploded view of port body 301. FIGS. 7A-7B illustrate respectively top views of vascular access port 300 in a delivery configuration and a deployed configuration, and FIGS. 8A-8B illustrate respectively top view and axonometric view of an exemplary arm 330 of vascular access port 300, in accordance with some embodiments.

Port body 301 is coupled with a septum member 302 covering a cavity 303 defined by the port body 301, and is configured for accepting a needle, for accumulating a dose of fluid, and for delivering fluid to or from vasculature via a catheter. Septum member 302, as well as cross section of cavity 303 are optionally generally elliptical, although they can be configured in other shapes. Port body 301 is assembled from a first structure comprising an inferior portion 313 that is connected (e.g., bonded and/or mechanically fastened) over a second structure comprising a posterior portion 314, as shown in FIG. 6C.

Posterior portion 314 is formed as a cap member opened at its bottom with generally ellipsoidal contour formed by front, rear and side walls thereof, and it comprises a top covering enclosing a cap opening 326 for allowing fluid delivery into or from cavity 303. Posterior portion 314 is connected to septum member 302 which seals cap opening 326 therefore restricting fluid administration to cavity 303 only by penetrating through septum member 302 with a needle connected to a fluid source (e.g., syringe).

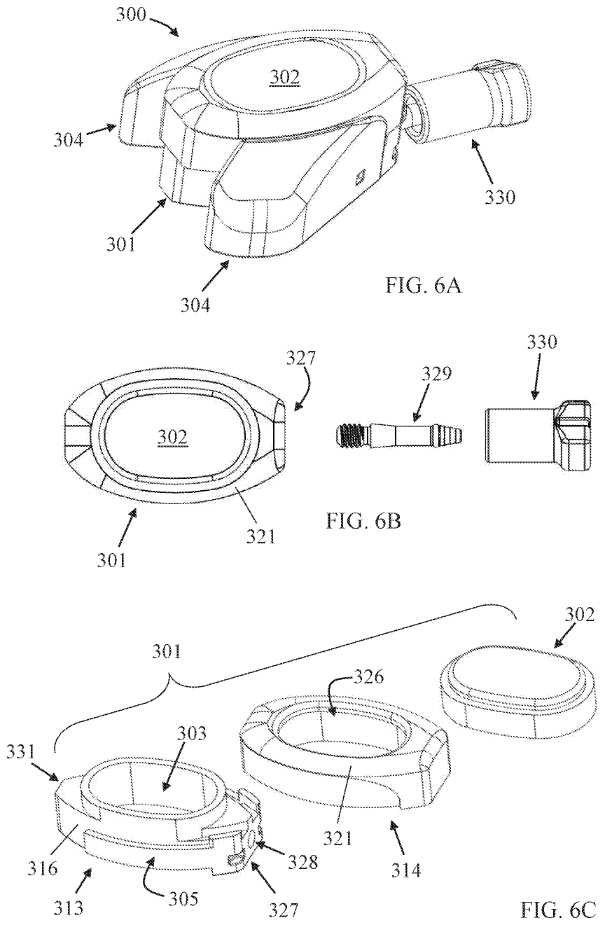

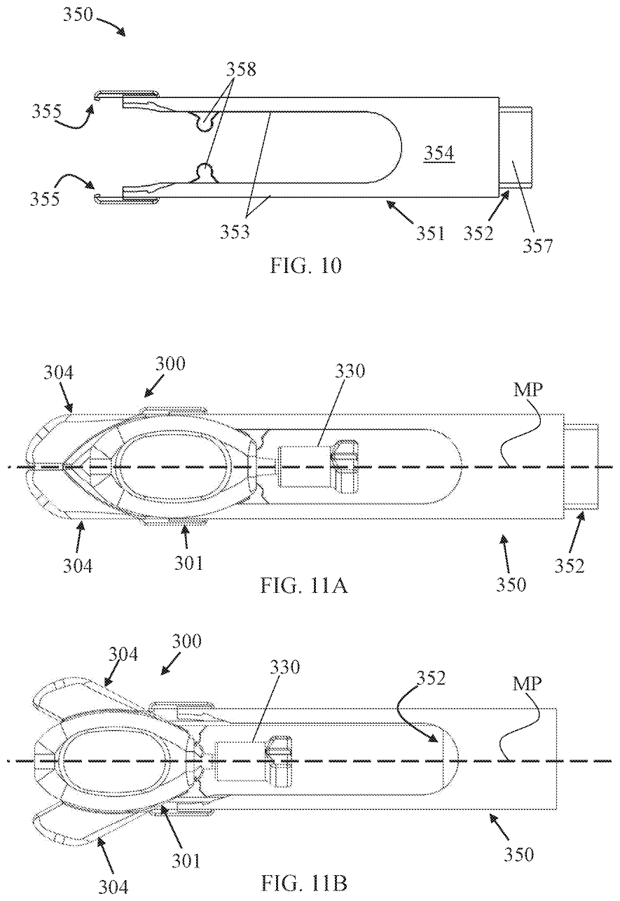

Inferior portion 313 includes a base 315 (Shown in FIG. 12A, for example) at its bottom and a wall 316 surrounding base 315 and cavity 303 below septum member 302. Wall 316 includes a front (distal) end 331, and a rear (proximal) end 327 with a base opening 328. Rear end 327 is connected to a catheter port 329 with a catheter connector 330 that passes through base opening 328 and configured for connecting to a proximal end of a catheter and establishing sealed fluid communication between cavity 303 and catheter lumen. On the outer surface of wall 316, periphery of inferior portion 313 includes a first lateral surface 317 spanning most or all right side thereof and a second lateral surface 318 spanning most or all left side thereof (as shown for example in FIG. 9A). Inferior portion 313 may be oval, elliptical, subelliptical, pyriform or vesica piscis shaped along the first and second lateral surfaces 317 and 318. Each one of first and second lateral surfaces, 317 and 318, is curved and has a constant radius of curvature being substantially greater than distance between opposing vertexes thereof, 319 and 320, respectively. Posterior portion 314 includes a brim-like rim 321 projecting laterally outwardly above the first and second lateral surfaces, 317 and 318, that is configured to cover a seam line formed between port body 301 and port body extension 304 when in the deployed configuration.

Port body extensions 304 are configured to stabilize and/or fixate port body 301 in-place in target implantation site IMS in the subject body when vascular access port 300 changes to the deployed configuration. Although port body 301 is configured to function completely as vascular access port, it may lack shape and size for proper stabilization in subcutaneous void VBS. It may also be more difficult for currently trained medical practitioners to locate port body 301 alone through skin tissues for accessing the port and administrating fluid therethrough, since that current practice involves use of substantially larger, generally rectangular shaped ports. When in the deployed configuration, port body extensions 304 add volume and provide a final shape for vascular access port 300 thereby improving stabilization and discovery. The total volume of the port body extensions 304, added to port body 301 in the formation of the deployed vascular access port 300, is at least about 15%, optionally at least about 30%, optionally at least about 50%, or optionally at least about 100% of the total volume of port body 301 alone.

Port body extensions 304 are restrictedly movable along respective defined routes 360 located on and along sides of port body 301, so as to facilitate the ability for selectively changing toggling vascular access port 300 from a delivery configuration to a deployed configuration by moving port body extensions 304 along defined routes 360. In some embodiments, port body 301 is configured to move axially (e.g., pushed distally) towards port body extensions 304 when changing to the deployed configuration, or alternatively, port body extensions 304 are moved axially (e.g., pulled proximally) towards port body 301, or any combination thereof. Port body 301 is configured to interlock with port body extensions 304 for forming vascular access port 300 as a unified structure in its final deployed shape and size. Port body 301 and port body extensions 304 are configured to change relative positioning therebetween when the vascular access port changes to the deployed configuration although each one of port body 301 and port body extensions 304 is configured to maintain size and shape thereof, and are optionally formed at least mostly from relatively rigid materials (besides at least septum member 302, for example, which is formed of flexible membrane).

In some embodiments, the defined routes 650 are parallel to a longitudinal axis of the port body coinciding with median plane MP so as to maintain a fixed distance between bottom (e.g., base 315) of port body 301 and a bottom 362 of each port body extension 304 when changing from the delivery configuration to the deployed configuration. In a case where bottoms of port body 301 and of port body extension 304 include planar bottom surfaces, as shown in FIGS. 12A and 12B for example, the planar bottom surface of port body 301 and planar bottom surfaces of port body extensions 304 are substantially coplanar and parallel to each other when in the delivery configuration and remain substantially coplanar when changing to and when in the deployed configuration.

Port body extensions 304 may be inserted into subcutaneous void SBV attached to or separately from port body 301, when the vascular access port 300 is in the delivery configuration, and to force increase volume enclosed by the subcutaneous void SBV by expanding therein when the vascular access port 300 changes to the deployed configuration, thereby outwardly compacting surrounding soft tissues. Port body extensions 304, collectively and/or separately, includes a front edge 333 pointing distally away from port body 301 and configured to cause atraumatic separation of tissue layers when forced to pass therebetween.

Port body extensions 304 include each an inner surface 325 (shown in FIG. 8B, for example) configured to cover front end 331 when in the delivery configuration and to cover the respective first or second lateral surface, 317 or 318, of inferior portion 313, when vascular access port 300 is in the deployed configuration, such that both port body extensions 304 collectively envelope some or most of wall 316 but without covering most or all rear end 327 and/or of front end 331. When changing from the delivery configuration to the deployed configuration, each one of port body extensions 304 rotates around an axis of rotation 361 and slides with inner surface 325 thereof on the respective one of two opposing sides (e.g., on lateral surface 317 or 318) of inferior portion 313. Axis of rotation 361 is located farther than and beyond a closer side of inferior portion 313 relative to the respective port body extension inner surface 325. As shown in FIGS. 7A and 7B for example, port body extensions 304 include a first arm 306 located right to a median plane MP of port body 301 and a second arm 307 located left to median plane MP. As shown in FIG. 8A for example, each one of first and second arms, 306 and 307, includes a wide head (front) portion 308 and a narrow body (rear) portion 309. When in the delivery configuration, head portions 308 are positioned axially distally to port body 301 and are in contact with each other along median plane MP, whereas body portions 309 surround distal portion of port body 301 from both sides of the median plane MP. When in the deployed configuration, head portions are 308 in juxtaposition from both sides of port body 301, each forming a gap 310 for allowing tissue ingrowth therebetween.

Toggle anchoring mechanism 305 can be selectively applied by a user (e.g., medical practitioner) by approximating the port body extensions 304 and port body 301 along median plane MP, and parting the laterally opposing first and second arms, 306 and 307, transversely to the median plane MP, thereby reducing length-to-width ratio of vascular access port 300. This way, head portions 308 are separated from each other transversely to median plane MP when shifting from the delivery configuration. When in the deployed configuration, port body extensions 304 are fixedly connected to port body 301 using a snap locking member 311 located at each proximal end of arm's body portions 309 that engages and locked into mating recess 312 at a proximal portion of port body 301, as shown in FIGS. 9B and 9C, for example. Locking member 311 can be released by the user, either manually or by using an instrument, for allowing selective reverting from the deployed configuration back to the delivery configuration for example.

First arm 306 is slidably connected to first lateral surface 317 and restrictedly movable along a first of defined routes 360, and second arm 307 is slidably connected to second lateral surface 318 and restrictedly movable along a second of defined routes 360. FIGS. 9A-9C illustrate axonometric views of exemplary inferior portion without (FIG. 9A) and with left arm of toggling vascular access port 300 in a delivery configuration (FIG. 9B) and a deployed configuration (FIG. 9C). As shown, toggle anchoring mechanism 305 is provided along each side of inferior portion 313 and includes a rail mechanism 322 extending along a length of the respective first or second lateral surface, 317 or 318, configured to restrict a defined rout of each one of the first and second arms, 306 and 307, along the respective first or second lateral surface, 317 and 318. Rail mechanism 322 includes a pair of geometrically mating curved elongated ridge 323 and groove 324, that are longitudinally interengaging with each other, one of them (e.g., ridge 323, as shown in FIG. 9A, for example) extends along the respective first or second lateral surface 317 or 318 and the other (e.g., groove 324, as shown in FIG. 8B, for example) extends along the respective first or second arm 306 or 307. Port 300 with toggle anchoring mechanism 305 are configured such that, when in the deployed configuration, first arm 306 covers (fully or mostly) the first lateral surface 317 and second arm 307 covers (fully or mostly) the second lateral surface 318, with inner surface 325 of each respective first or second arm 307 or 308 is in contact with the respective first or second lateral surface, 317 or 318, with substantially no gap therebetween.

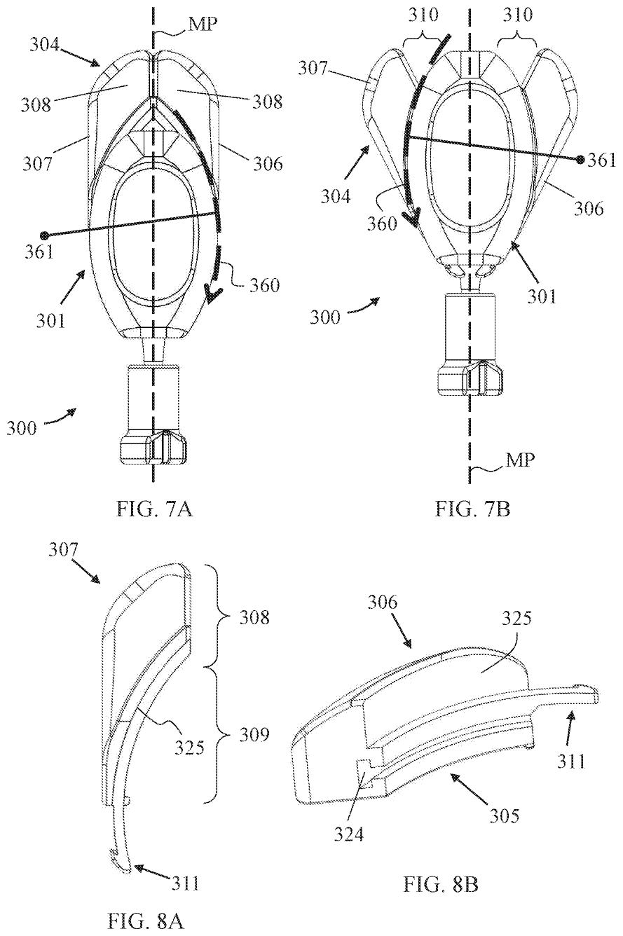

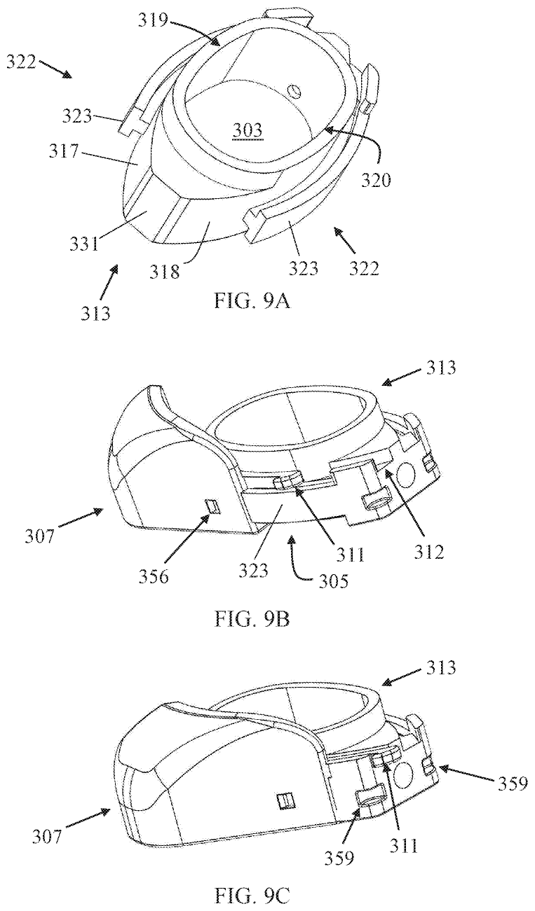

Reference is now made to FIG. 10 which illustrates top view of an exemplary delivery apparatus 350 of toggling vascular access port 300. Delivery apparatus 350 includes a port gripping member 351 connected to and axially slidable over a port pushing member 352. Port gripping member 351 is configured for gripping port body extensions 304 during port 300 delivery to target implantation site IMS and during transitioning from the delivery configuration to the deployed configuration in implantation site IMS. Port gripping member 351 includes two elongated gripping member arms 353 extending axially with gap therebetween sufficient for accommodating catheter connector 330 (as shown in FIG. 11A, for example) and optionally also a proximal portion of a catheter connected thereto. The two gripping member arms 353 are adjoined at a proximal portion 354 of port gripping member 351 which is optionally also configured for manual grasping and/or maneuvering by a user. Each gripping member arm 353 includes a gripping portion 355 at distal tip thereof configured for engaging a mating gripping recess 356 at the outer side of the respective port body extension 304 (shown in FIG. 9B, for example).

Port pushing member 352 is configured for axially translating (moving) port body 301 relatively to port body extensions 304 during transitioning from the delivery configuration to the deployed configuration in implantation site IMS. Port pushing member 352 includes two elongated pushing member arms sliding within gripping member arms 353. The two pushing member arms are adjoined at a proximal portion 357 of port pushing member 352 which is optionally also configured for manual grasping and/or maneuvering by a user. Each pushing member arm includes a pusher portion 358 at distal tip thereof configured for engaging a mating pusher recess 359 at the rear end 327 on inferior portion 313 of port body 301 (shown in FIG. 9C, for example). Once in the subcutaneous void or passage SBV, the user can either push proximal portion 357 of port pushing member 352, while holding in-place proximal portion 354 of port gripping member 351, for pushing port body 301 distally for shifting to the deployed configuration (similarly to as shown in FIG. 5B, for example); or pull proximal portion 354 of port gripping member 351, while holding in-place proximal portion 357 of port pushing member 352, for pulling port body extensions 304 proximally for shifting to the deployed configuration (similarly to as shown in FIG. 5C, for example), or any combination thereof.

FIGS. 11A-11B illustrate top views of delivery apparatus 350 equipped with toggling vascular access port 300 in a delivery configuration and a deployed configuration, respectively. FIGS. 12A-12C illustrate axonometric views of a portion of delivery apparatus 350 demonstrating use of port gripping member 351. The user (e.g., medical practitioner) may receive vascular access port 300 already assembled with delivery apparatus 350 as shown in FIG. 11A, or the two devices should first be assembled after unpackaging. Delivery apparatus 350 may be provided in same package with port 300 as a kit, or they may be provided in separate packages. Optionally, a user can first determine if a specific type or variation of port 300 is needed, optionally of a particular size, shape of the septum member, or otherwise and then connect it to delivery apparatus 350. Similarly, delivery apparatus 350 may be provided in different sizes (e.g., lengths) or in one size for one or different variations of port 300.

After toggling vascular access port 300 is properly connected to delivery apparatus 350, it can then be inserted via a previously formed surgical opening through skin tissue layers to subcutaneous target implantation site IMS in the subject body, when it is in the delivery configuration. Subcutaneous void or passage SBV may be formed or be part of a subcutaneous surgical tunnel being at least 4 cm , or at least 6 cm, or at least 10 cm long, and which may be formed either by front ends of port 300 (of port body extensions 304 particularly) when in the delivery configuration, by a front end of delivery apparatus 350, or by a separate surgical tunneler, for example. Vascular access port 300 may be already connected to a catheter when provided to the user, or the user has to connect the two either before or after implantation of at least one of port 300 in target site IMS, or the catheter in the subject's target vasculature.

Once in position, port 300 can then be changed into the deployed configuration (FIG. 11B) by approximating port body extensions 304 to port body 301 along median plane MP, which also applies toggle anchoring mechanism 305 to push port body extensions 304 transversely to the median plane, thereby reducing length-to-width ratio of vascular access port 300 for anchoring it in the target implantation site IMS. The anchoring is met by at least pressing against surrounding tissues generally away from the median plane MP. As described above, such approximation can be achieved for example by holding port body extensions 304 in place with port gripping member 351 and pushing port body 301 distally with port pushing member 352, or vice versa.

Shifting to the deployed configuration may also cause automatic detachment of the properly deployed port 300 from delivery apparatus 350, as demonstrated in FIGS. 12A-12C. Gripping portions 355 are configured to hold port 300 by protruding laterally inwardly in gripping recesses 356 and/or clinging thereto by applying force generally towards their respective pusher portions 358 which provide a counter force for gripping the port 300 therebetween. Port body extensions 304 and toggle anchoring mechanism 305 are configured such that gripping recesses 356 remain substantially fixed in place while port body extensions 304 revolve around sides of port body 301 when shifting from the delivery configuration to the deployed configuration. In some embodiments, only when port body extensions 304 interlock with port body 301, when locking members 311 properly engage and locked into respective recesses 312, at least one of gripping recesses 356 shifts and/or revolves relative to its respective gripping portion 355 sufficiently for allowing detachment therefrom, as shown in FIG. 12C. this design also serves as safety mechanism that assures release from port 300 only when it is properly deployed and locked in its final shape.

After toggling vascular access port 300 is released, delivery apparatus 350 is removed from the subcutaneous void or passage VBS, and the surgical opening can be closed (e.g., by suturing). Port 300 can be removed from the body, either immediately or after prolonged use (weeks or months, for example) by surgically accessing rear end 327 of inferior portion 313, releasing locking members 311 from recesses 312, reversing toggle anchoring mechanism 305 to change port 300 back to the delivery configuration, and then pulling port 300 from the implantation site IMS. The unique design of vascular access port 300 allows pushing the tissues grown distally thereto while reducing in thickness, such that it can be easily removed with minimal to no soft tissue entrapment between the port parts during and after changing it back to the delivery configuration.

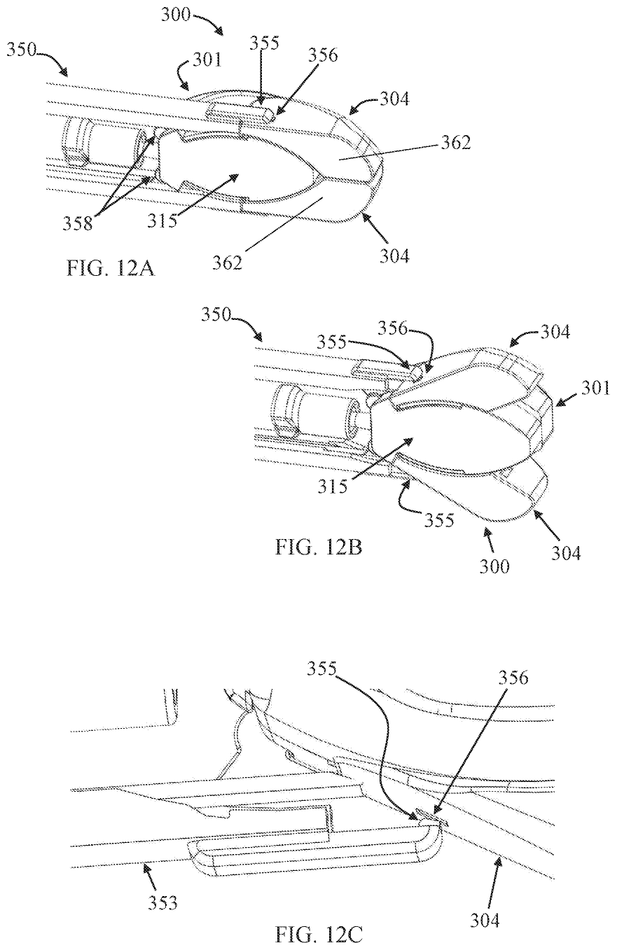

FIGS. 13A-13C illustrate axonometric views of another exemplary toggling vascular access port 400 which is an exemplary variation of toggling vascular access port 300 having similar or identical features, except for a few other (e.g., additional or alternative) features such as: (a) a structural and/or functional feature allowing delivering and/or deploying of port 400 using medical forceps or other grasping capable instrumentation instead or in addition to dedicated delivery devices connectable thereto such as delivery apparatus 350, and/or (b) a structural and/or functional feature allowing automatic change in total height of port 400 when shifting from the delivery to the deployed configuration.

Similarly to port 300, toggling vascular access port 400 includes a port body 401 and at least one port body extension 404 restrictedly movable along an at least one defined route 405 on port body 401. The at least one port body extension 404 includes a first arm 408 located right to a median plane of port body 401 (similar to median plane MP shown in FIGS. 7A and 7B, for example) and a second arm 409 located left to the median plane.

Port body extensions 404, particularly first and second arms 408 and 409, are each rotatably and slidably connected to port body 401 and configured to rotate around an axis of rotation and slide on an at least one of two opposing sides of the port body 401, along routes 405, when changing from the delivery configuration to the deployed configuration. Port body 401 has an inferior portion 410 and a posterior portion 411, the posterior portion 411 is connected to a septum member 402 and the inferior portion 410 surrounds a cavity 403 that is defined by port body 401 and located below and covered by septum member 402. Inferior portion also includes a first lateral surface spanning most or all right side of inferior portion 410 and a second lateral surface spanning most or all left side of the inferior portion 410. A rear end 414 of port body 401 is coupled to a catheter connector 415 configured for connecting to a proximal end of a catheter (e.g., catheter 15 shown in FIG. 1) for facilitating fluid communication between cavity 403 and a lumen of the catheter.

Toggling vascular access port 400 is selectively changeable from a delivery configuration (shown in FIG. 13A, for example) to a deployed configuration (shown in FIG. 13B, for example) by moving first and second arms 408 and 409 along respectively along a first and a second of routs 405. When in the delivery configuration, a front portion 407 of each port body extension 404 is positioned axially distally to the port body 401. When changing to the deployed configuration, port body extensions 404 and the port body 401 are approximated along the median plane of port body 401 coincidently with laterally opposing portions 406 of port body extension 404 being parted transversely to the median plane, thereby reducing length-to-width ratio of the toggling vascular access port 400. When in the deployed configuration, the port body extensions 404 are fixedly and releasably connected to port body 401, therefore allowing selective reverting from the deployed configuration to the delivery configuration. Furthermore, rear end 414 of port body 401 is kept not covered with the port body extensions 404 also after changing to the deployed configuration, for avoiding engagement with catheter connector 415 and/or a catheter connected thereto, for example.

A port clamping portion 416 is located on the ear end 414 of port body 401 superiorly to catheter connector 415 for allowing a user to selectively move and/or manipulate port 400 subcutaneously and in the target implantation site while avoiding engagement with catheter connector 415 and/or a catheter connected thereto, for example. A user can clamp port clamping portion 416 with medical forceps and push toggling vascular access port 400 when in the delivery configuration to the target implantation site with the medical forceps. Once in the target implantation site, port 400 can be changed to the deployed configuration by pushing port body 401 distally relative to port body extensions 404 and/or pulling port body extensions 404, such as with pulling members 419 connected to first and second arms 408 and 409 while resisting motion of port body 401 using the forceps.

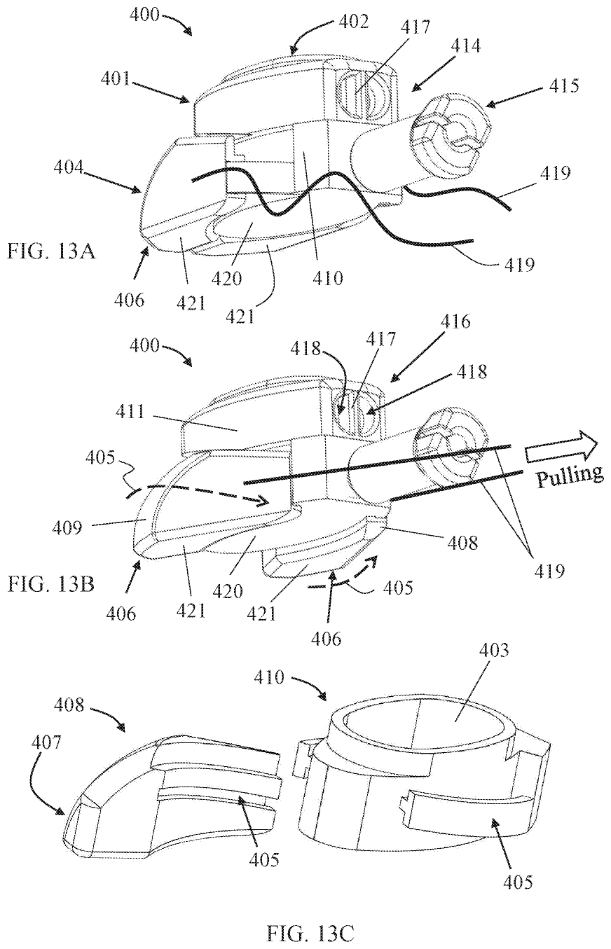

Port clamping portion 416 includes a thin wall portion 417 comprising opposing lateral surfaces extending parallel to the median plane from both sides thereof, the wall portion 417 is configured for grasping and/or clamping by medical forceps including but not limited to needle holder or Kelly forceps 430, as shown in FIGS. 14A and 14B. In some embodiments, wall portion 417 is about 0.5 mm to 3 mm (optionally particularly about 1 mm to 2 mm) thick and/or about 2 mm to 5 mm (optionally particularly about 3 mm to 4 mm) wide for allowing sufficient clamping contact area and buildup of sufficient clamping, grasping or locking force from both sides of wall portion 417 using medical forceps.

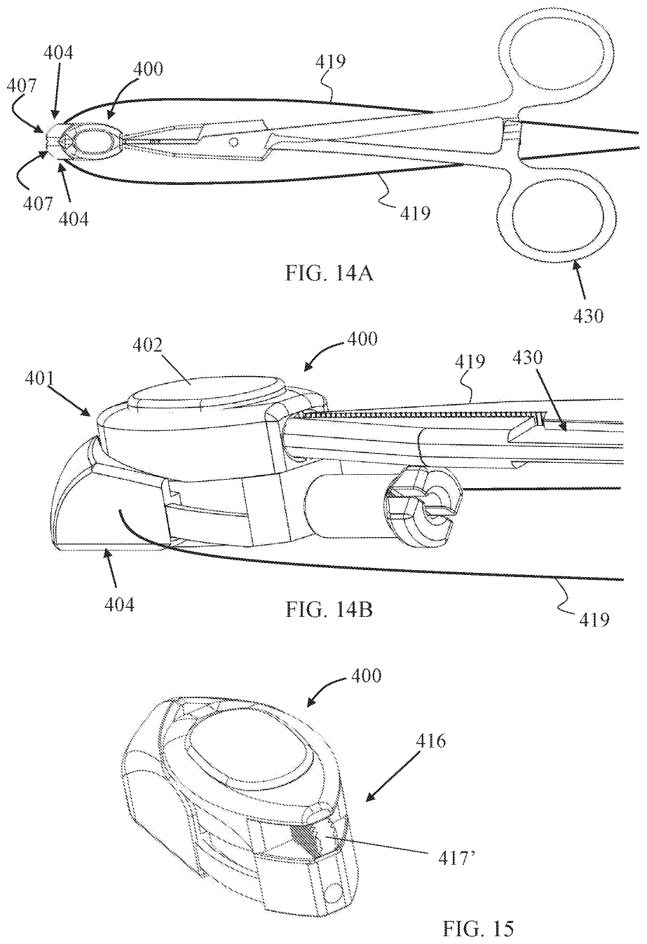

Wall portion 417 can be configured as a septum dividing cavities 418 formed in rear end 414 from both sides thereof. Cavities 418 are shaped and sized to accommodate a pair of tips of the medical forceps and to allow closing motion of the pair of tips thereinside towards the wall portion and grasping of the wall portion 417 with the pair of tips from both sides thereof. FIG. 15 illustrates an alternative exemplary configuration of port clamping portion 416 having a thin wall portion 417′ similar to wall portion 417, yet not bound by cavities, rather allow greater room for forceps tips maneuverability.

In some embodiments, a planar bottom surface 420 of port body 401 is substantially coplanar with respective planar bottom surfaces 421 of port body extensions 404 only when toggling vascular access port 400 is in the delivery configuration, but are not coplanar when in the deployed configuration. In some embodiments, each one of the defined routs 405 is inclined to the longitudinal axis of port body 401 so as to gradually increase or decrease distance between bottom surface 420 of port body 401 and bottom surfaces 421 of port body extensions 404, such that they optionally diverge from being coplanar, when changing from the delivery configuration to the deployed configuration. Nevertheless, planar bottom surface 420 and planar bottom surfaces 421 may be configured to remain substantially parallel to each other when changing from the delivery configuration to the deployed configuration.

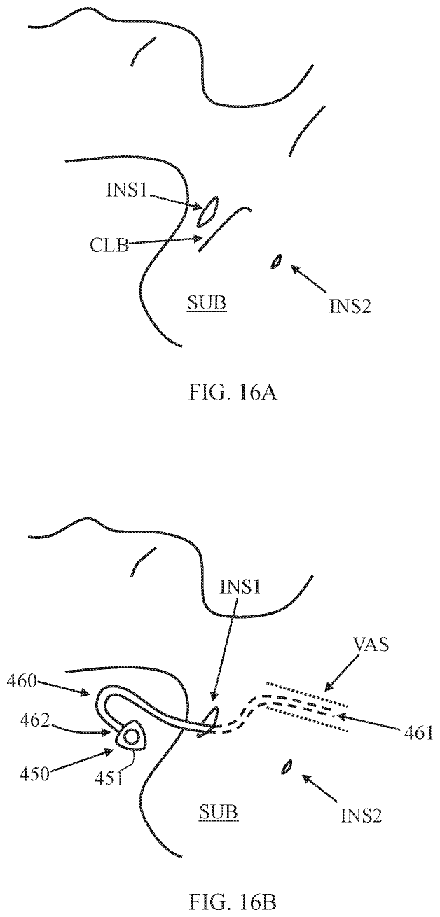

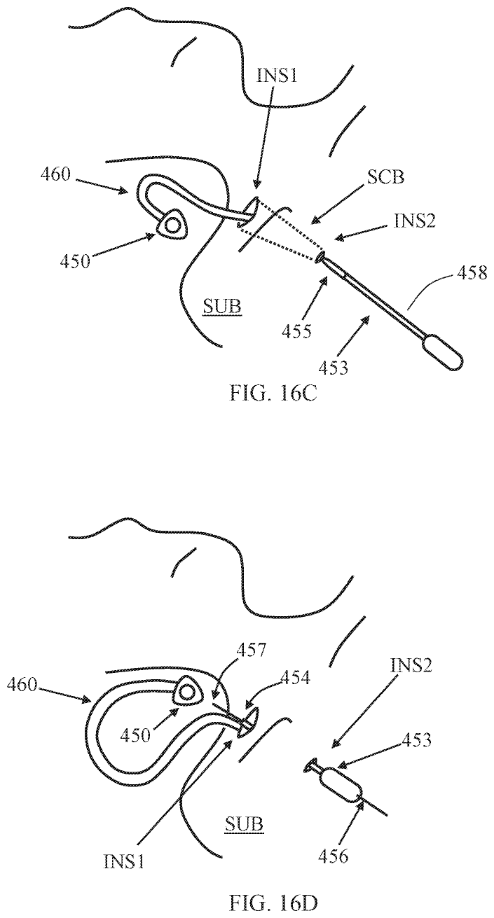

Reference is made to FIGS. 16A-16H which schematically illustrate several views representing possible scenarios in execution of a method for subcutaneously delivering and implanting a vascular access port 450, which may result in part or in full, in several aspects, with implantation of port 10 shown in FIGS. 1A-1B. Vascular access port 450 may be similar, identical or equivalent-structurally, functionally and/or dimensionally—to either one of port 10, port 20, port 60, port 100, port 300 and port 400, and includes a port body 451 coupled to a septum member (e.g., septum member 502 shown in FIG. 17D, for example) covering a cavity (e.g., cavity 503 shown in FIG. 17D, for example) defined by port body 451. Port body 451 is optionally connectable to or includes an expandable element configured to change shape, or increase size, area and/or volume of vascular access port 450 around the septum member when expanded or otherwise changing in shape, for allowing passing of port 450 subcutaneously in a relatively small size, and increasing its size after it is positioned at the implantation site. Optionally, alternatively or additionally, vascular access port 450 is connectable to a port body extension (as will be described with respect to vascular access port 500) to form a unified structure having a final shape and size. In some embodiments, the final shape of vascular access port 450, if as a unified structure or in its basic structure, is generally triangular as defined by side periphery thereof.

As shown in FIG. 16A, a first incision INS1 is formed to a skin layer of subject's body SUB, optionally superiorly to (above) the right collarbone CLB, and an entry point (optionally, a second incision) INS2 is made inferiorly to (below) first incision INS1 in the chest area below collarbone CLB. In some embodiments, first incision INS1 is greater in size (length) than second incision INS2, such that it is sized to allow entering of vascular access port 450 therethrough, while second incision INS2 is sized to prevent or at least resist travel of vascular access port 450 therethrough. Each one of incisions INS1 and INS2 can be made with a sharp object such as by way of cutting (e.g., using a scalpel for example) or puncturing (e.g., using a pointed object). Second incision INS2 can be formed before or after first incision INS2 is made and at any stage of the procedure. Alternatively, only first incision INS1 is made without any other incision throughout the entire procedure. A catheter 460 is introduced into a vascular system VAS of subject's body SUB via first incision INS1 (FIG. 16B), such that a distal end 461 of catheter 460 is positioned in a target blood vessel, for example the superior vena cava. A proximal end 462 of catheter 460 can be provided readily affixed to vascular access port 450 and having a lumen thereof in fluid communication with the port cavity; or alternatively it can be provided disconnected from port 450 and is later connected to vascular access port 450 thereby forming fluid communication between a lumen of catheter 460 and the port cavity.

A subcutaneous passage SCB is created between an entry point INS2 to first incision INS1, by pushing an elongated introducer 453 from entry point INS2 (FIG. 16C) to first incision INS1 until a distal end 454 of introducer 453 emerges from within the skin layer proximately to first incision INS1, as shown in FIG. 16D. Optionally and alternatively, subcutaneous passage SCB is created by pushing and/or withdrawing with a separate surgical tunneler before passing introducer 453 therethrough.

In some embodiments, introducer distal end 454 is pushed from entry point INS2 towards first incision INS1 to form subcutaneous passage SCB while it is covered with a cover 455 having a pointed or rounded front. After introducer distal end 454 protrudes via first incision INS1, cover 455 is removed for allowing port 450 to be coupled to introducer 453. The length of subcutaneous passage SCB, which is about the distance from entry point INS2 to first incision INS1, is substantially greater than maximal dimension (e.g., length) of vascular access port 450, and is optionally equal to or greater than 5 cm.

Vascular access port 450 is then coupled to introducer 453 (FIG. 16E) and withdrawn via first incision INS1 until it is fully implanted in subcutaneous passage SCB proximately to entry point INS2 (FIG. 16F). In some embodiments and as shown, port 450 is greater in width from the subcutaneous passage SCB originally formed with cover 455 so by pulling port 450 with introducer 453 towards entry point INS2 it forces subcutaneous passage SCB to widen. In some embodiments, port 450 is coupled to introducer 453 with a shaft 456, by connecting to a distal end 457 of shaft 456 when it is protruding from within a tubular body 458 of introducer 453. Optionally, vascular access port 450 is connected by fastening to a shaft fastener provided at the shaft distal end 457 optionally by using male and female fastening mechanism. Before coupling, shaft 456 can be fixated to introducer 453 such as by connecting proximal ends thereof. Once the physician concludes the procedure has completed, introducer 453 and shaft 456 are uncoupled from port 450 and removed (FIG. 16G) and incisions INS1 and INS2 are closed with sutures (FIG. 16H).

FIGS. 17A-17D illustrate an exemplary vascular access port 500 before (FIGS. 17A & 17B) and after (FIGS. 17C & 17D) interlocking with an exemplary port body extension 530. Vascular access port 500 may be considered an exemplary variation of port 450 and/or may be similar, identical or equivalent-structurally, functionally and/or dimensionally—to either one of port 10, port 20, port 60, port 100, port 200, port 300, port 400 and port 450. Port 500 includes a port body 501 coupled to a septum member 502 covering a cavity 503 defined by port body 501. The materials of the port body 501 can be metal, polymer or metal and polymer combined. The septum member 502 is a silicon septum that can withstand 2,000 punctures or more of a non-coring needle without failing. cavity 503 is surrounded with thin walls and base.

Port 500 also includes a first connector 505 releasably connectable to a shaft fastener 581 (shown in FIG. 17B, for example), and a second connector 507 enclosing a connector lumen 508 configured to facilitate fluid communication between cavity 503 and a lumen of a catheter (such as catheter 460, for example), when a proximal end of the catheter is connected thereto. Port body 501 has a generally triangular shape, at least in front portion thereof, defined by side periphery 509 thereof, wherein first connector 505 is affixed to and protrudes adjacent to or at the center of a vertex 510 of port body 501, and second connector 507 is affixed to, and protrudes adjacent to or at the center of a rear side 511 of port body 501 opposing port body vertex 510. Second port body connector 507 is optionally provided detached from a catheter (as shown in FIG. 17C, for example), or is provided readily connected to the catheter (as shown in FIG. 16B, for example).

In some embodiments, the size and/or shape of port body 501 is designed particularly for improved maneuverability and/or for allowing passing under acceptable resistance through a subcutaneous passage (optionally, while slightly widening it), such as percutaneous passage SCB shown in FIG. 16D for example, yet it is not configured or is less efficient for long term implantation and use as required from subcutaneously implanted vascular access ports. In some such embodiments, port body 501 is configured for interlocking with port body extension 530 to form a unified structure 540 having a final shape and size which are preferred for long-term implantation and use. Port body 501 and/or port body extension 530 may include rails 535 to guide port body extension 530 for properly engaging and interlocking to port body 501. Unified structure 540 is wider than vascular access port 500 and maintains a generally triangular shape defined by side periphery thereof.

A vertex 541 of unified structure 540 includes a third connector 542 releasably connectable to an introducer fastener 571 (shown in FIG. 17B, for example). A first portion 531 of port body extension 530 is connectable to a first side 512 of vascular access port 500, and a second portion 532 of port body extension 530 is connectable to a second side 513 of vascular access port 500. First portion 531 and second portion 532 may be part of or be in a form of two arm pivotally or flexibly connected via unified structure vertex 541.

Optionally, port body extension 530 is provided in a collapsed configuration, in which first and second portions 531 and 532 are in proximity or even in contact with each other, such that its maximal dimension approximates to (e.g., equal to or smaller than) size of the subcutaneous passage or to maximal dimension of port body 501. Optionally, port body extension 530 is expandable, optionally self-expandable, from the collapsed configuration to an opened configuration (illustrated in FIG. 17A, for example), in which first and second portions 531 and 532 are distant one to the other, such that the distance formed between them is sufficient to accept first and second sides 512 and 513 of port 500 thereinside and to facilitate interlocking thereof with first and second portions 531 and 532. Interlocking is possible optionally by snap locking each portion of port body extension 530 with its corresponding side of port 500. Following interlocking, rear side 511 of port body 501 remains uncovered by any portion of port body extension 530. The materials of the port body extension 530 can be metal, polymer or metal and polymer combined. The rails of port body extension 530 are designed to reduce the gaps after interlocking with port body 500, by that achieving less voids for infection nucleus. The port body extension is design in a manner that the un-collapsed shape is predetermined. port body extension 530 when collapsed is with the same frontal cross section dimensions such as of the port body or less.

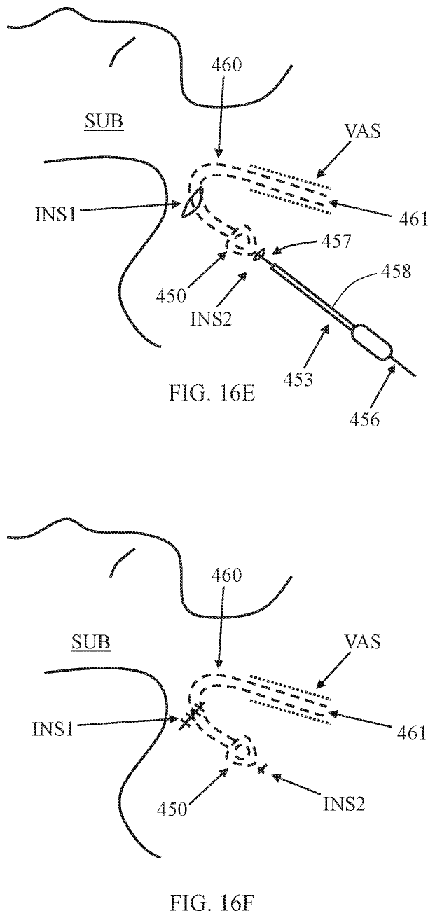

FIGS. 18A-18D illustrate several views representing possible scenarios in execution of a method for deploying vascular access port 500, using an exemplary delivery device 560. Delivery device 560 may be provided in a kit that comprises at least two of: the port 500, a catheter (e.g., catheter 460) optionally readily connected to port 500; delivery device 560 comprises of an elongated introducer 570 and an inner shaft 580 connectable to each other.

Elongated introducer 570 has a tubular body 572 having a distal end 573 provided covered with a removable cover 574 (FIG. 18A) optionally fixated to introducer 570 by way of threading. Cover 574 is dimensionally, structurally and/or functionally similar or identical to front ends of exemplary known surgical tunnelers and has a pointed or rounded front 575 configured for forming surgical subcutaneous passage when pushed beneath skin layer using a handle 576 which is located on proximal end 577 of introducer body 572.

FIG. 18B shows inner shaft 580 provided with a handle 582 connected to handle 576 of introducer 570, and with distal end 583 thereof protruding from within introducer tubular body 572 (after cover 574 was removed). Also shown in FIG. 18B is port body extension 530 fastened to introducer fastener 571 provided at introducer distal end 573. Shaft 580 and port body extension 530 can be fixated to introducer 570 after it extends along subcutaneous passage with distal end 573 thereof protruding from the subcutaneous passage, as shown for example in FIG. 16D with respect to introducer distal end 454. A shown, port body extension 530 is in a collapsed configuration after it is connected to introducer 570 and before coupling with port 500, wherein first and second portions 531 and 532 thereof are in proximity with each other.

After connecting port body extension 530, vascular access port 500 is fastened to shaft fastener 581 that is provided at shaft distal end 583 protruding from within introducer 570 (FIG. 18C). In this configuration, port 500 and port body extension 530 can be pulled back together, disassembled and longitudinally spaced with each other, using delivery device 560, such that maximal dimension of each is sufficiently small to pass through the subcutaneous passage towards the target port implantation site adjacent one of the incisions (as shown in FIG. 16E, for example).

When port body extension 530 is positioned at the target implantation site, proximately to entry point of introducer 570 into the subcutaneous passage, inner shaft 580 is disconnected from introducer 570 (e.g., by way of separating, such as by loosening, shaft handle 582 from introducer handle 576) and is pulled distally relative to introducer 570 which is kept in-place. This way, vascular access port 500 is drawn towards port body extension 530 until they are engaging and connected with each other into forming unified structure 540, as shown in FIG. 18D. While engaging, port 500 forces first and second portions 531 and 532 to shift laterally away from each other, as described above. Once connected, unified structure 540 is uncoupled from delivery device 560 by unfastening port body extension 530 from introducer fastener 571 and unfastening vascular access port 500 from shaft fastener 581.

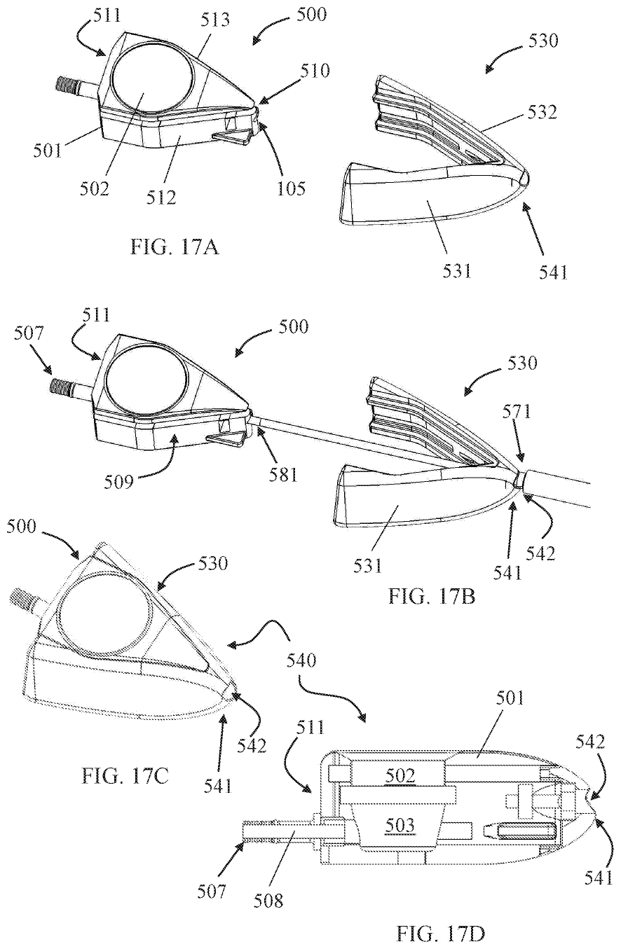

Reference is now made to FIGS. 19A-19D which schematically illustrate optional members (e.g., instruments, devices and/or components) in an exemplary kit 600 for deploying and implanting a vascular access port 601 using a surgical tunneler 602. Kit 600 may include surgical tunneler 602 shown in FIG. 19A. Kit 600 may include tunneler 602 and a port delivery apparatus 603 (shown in FIG. 19B), which may be similar structurally and/or functionally at least in part to delivery apparatus 350 or delivery device 560, for example. Kit 600 may also include vascular access port 601 (FIG. 19C) which may be similar structurally and/or functionally at least in part to one or more of port 10, port 20, port 60, implant 100, port 200, port 300, port 400 and port 450. Kit 600 may also include a catheter 604 shown in FIG. 19D (e.g., similar to catheter 15 or catheter 460).

The term “surgical tunneler” or “tunneler” described herein, and as known in the relevant art, refers to a surgical apparatus configured for forming an elongated subcutaneous passage between an entry point (e.g., incision) formed across skin tissue and a remote subcutaneous void and/or target implantation site. In some embodiments, surgical tunnelers are also configured for maintaining sufficient space along the subcutaneous passage by holding or outwardly compressing, with tubular wall thereof, soft tissues surrounding the subcutaneous passage, thereby allowing unhindered delivering and/or deploying vascular access ports (like port 601) and catheter connected thereto (e.g., catheter 604). Such delivery and/or deployment can be accomplished by using one or more portions, parts or components of the surgical tunneler itself or by using other dedicated instruments or devices, such as delivery apparatus 603, capable of passing through the lumen of the surgical tunneler into the subcutaneous void or target implantation site.

Surgical tunneler 602 includes a tubular tunneler body 605 being at least 4 cm, optionally at least 10 cm, in length, and enclosing a tunneler lumen 606 being a at least 0.5 cm, optionally at least 2 cm in diameter. Tunneler body 605 is connected at a distal end thereof to a tunneler tip 607 that is shaped and configured for forming subcutaneous tunnels, such as by way of dissection, when forcefully pushed beneath skin layers with surgical tunneler 602. Surgical tunneler 602 can be applied manually (e.g., pushed and/or manipulated) via a tunneler handle 608 connected to a proximal end of tunneler body 605.

Tunneler tip 607 is optionally configured for selective detachment or breaking from tunneler body 605 for implantation within the body such as within the subcutaneous void or target implantation site, following its use for tunneling when coupled with tunneler body 605. In some embodiments, tunneler tip 607 is formed separately or detached from tunneler body 605 as part of manufacturing of surgical tunneler 602 and, optionally, is afterwards releasably connected to distal end of tunneler body 605 before packaging of surgical tunneler 602. In some other embodiments, tunneler tip 607 is formed as an integral portion of tunneler body 605 and manufacturing of the whole part (combining tunneler tip 607 and tunneler body 605) may also include formation of weakening portions (lines and/or points, for example) at a chosen breaking portion configured for breaking under a force being greater than a predetermined magnitude and/or applied in a chosen direction, hence separating tunneler tip 607 from tunneler body 605 adjacent to the breaking portion.

Port delivery apparatus 603 includes an elongated delivery apparatus body 610 having sufficient length and rigidity to push and/or maneuver port 601 through and across tunneler lumen 606 via a proximal opening thereto. Delivery apparatus body 610 is connected to or adjoins a port connector 609 releasably connectable to a rear (proximal) side or portion of port 601. Port connector 609 may be controllable, at least for selectively release locking to port 601, from proximal end of delivery apparatus body 610. In some embodiments, port delivery apparatus 603 is further configured to facilitate selective detachment and/or breaking of tunneler tip 607 from tunneler body 605 such as by transferring sufficient force and/or by releasing a locking or connecting mechanism connecting tunneler tip 607 to tunneler body 605. In some such embodiments, port delivery apparatus 603 connected to port 601 can be applied to engage port 601 with tunneler tip 607, such as by way of pushing one against the other, so as to connect therebetween and then to detach or break tunneler tip 607 from tunneler body 605, thereby leaving a united structure 611 comprising of port 601 and tunneler tip 607 implanted in the body (e.g., in the subcutaneous void and/or target implantation site).

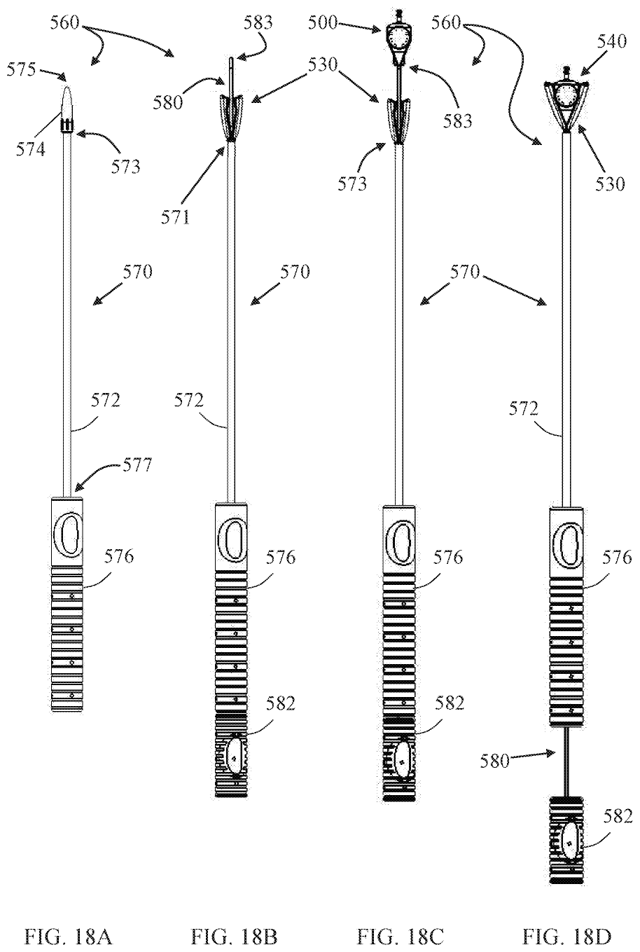

FIGS. 20A-20D schematically illustrate several views representing possible scenarios in execution of a method for subcutaneously delivering and implanting a vascular access port 601 using the exemplary kit 600, in accordance with some embodiments. As shown in FIG. 20A, surgical tunneler 602 equipped with tunneler tip 607 can be applied to form a subcutaneous surgical tunnel SST beneath skin tissues of a live subject SUB, until reaching or forming a subcutaneous void SBV in proximity or enclosing a target implantation site IMS. Port 601 connected to catheter 604 and held with port delivery apparatus 603 can then be inserted into tunneler lumen 606 from outside subject's SUB body, and advanced towards rear (proximal) end of tunneler tip 607. As shown in FIG. 20B, port delivery apparatus 603 can then be applied to connect between port 601 and tunneler tip 607 and force detachment or breaking of tunneler tip 607 from tunneler body 605 and place the unified port structure 611 in the target implantation site IMS. Port delivery apparatus 607 can then be released from port 601 and withdrawn from subject SUB via tunneler lumen 606 (FIG. 20C), leaving unified port structure 611 in target implantation site IMS with catheter 604 connected to port 601 and extending along tunneler lumen 606. Afterwards, surgical tunneler 602, detached from tunneler tip 607, can be withdrawn from subcutaneous tunnel SST and from the body of subject SUB, leaving unified port structure 611 in target implantation site IMS with catheter 604 connected to port 601 and extending along subcutaneous tunnel SST.

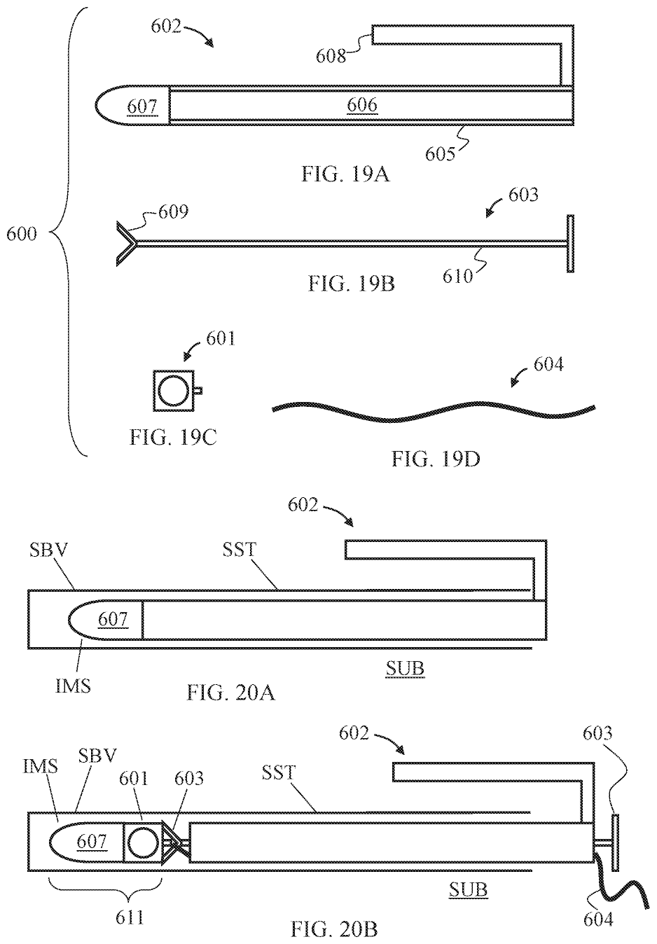

In some embodiments, the unified port structure 611 may be benefited from additional means for stabilization and/or anchoring in the subcutaneous void SBV, particularly if surgical tunneler 602 dimensions limits size and shape of tunneler tip 607 and/or port 601. FIG. 21A shows a first exemplary variation 611′ of unified port structure 611 in kit 600, in which port 602 includes releasable stabilizing legs 612 configured to flex outwardly relative to port 601, when upon release from tunneler lumen 606 or when selectively expanded by a user outside of tunneler lumen 606, in subcutaneous void SBV. FIG. 21B shows a second exemplary variation 611″ of unified port structure 611 in kit 600, in which tunneler tip 607 is expandable and/or shapeable when forced to engage with port 602, upon detachment or breaking from tunneler body 605 or when selectively expanded by a user when port 601 is outside of tunneler lumen 606, in subcutaneous void SBV.

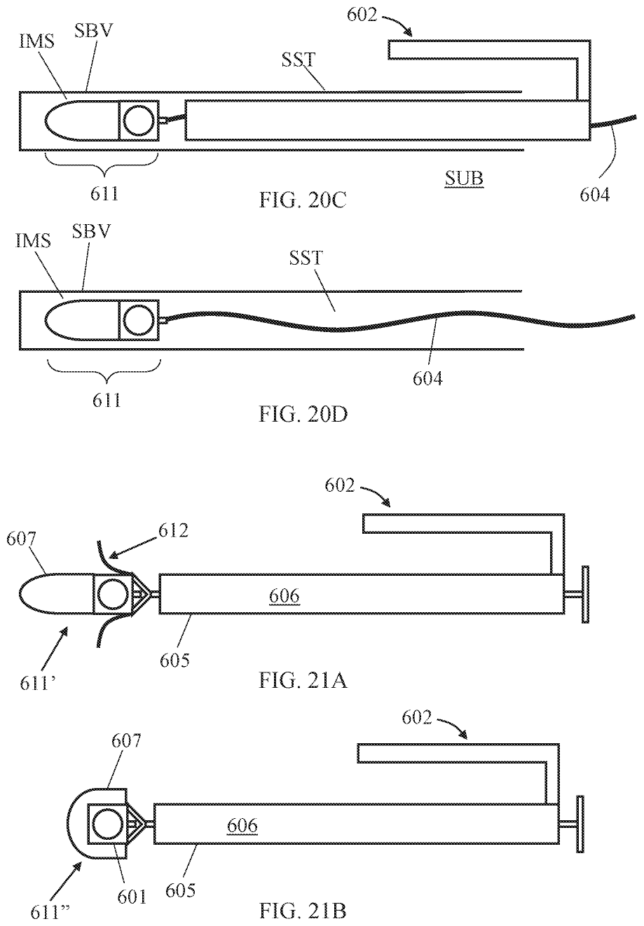

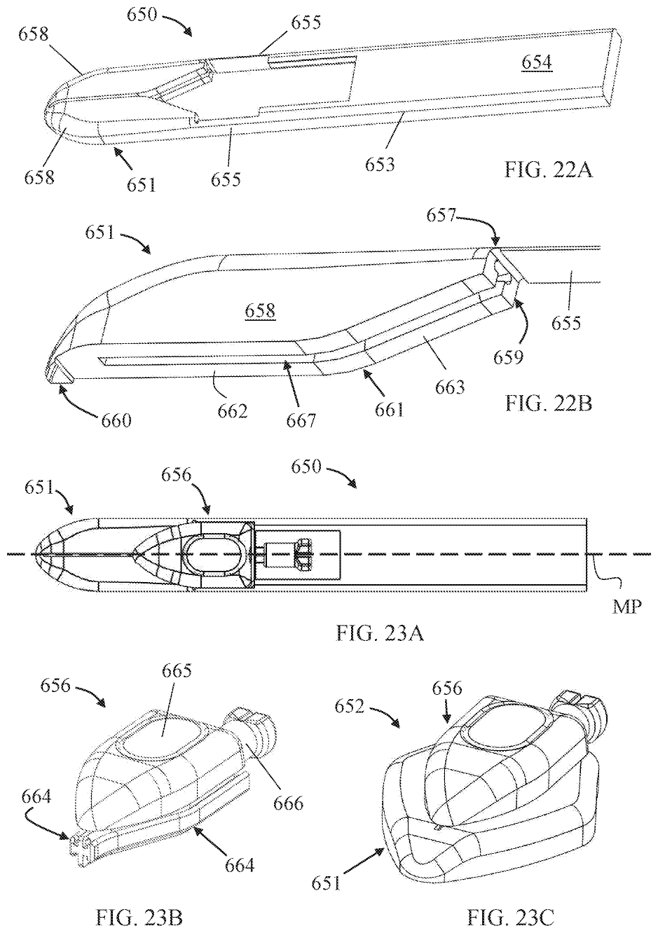

FIGS. 22A-22B illustrate respectively a full axonometric view and a partial cut axonometric view of an exemplary tunneling and port delivery apparatus 650 comprising an implantable tunneler tip 651. In some embodiments, apparatus 650 may be considered an exemplary variation of surgical tunneler 602 that integrates, and/or obviates separate use of, a delivery device such as port delivery apparatus 603, delivery apparatus 350, although it may require use of an additional device (e.g., medical forceps, or a rod-like member or probe for example) for deploying a vascular access port in a target implantation site. FIGS. 23A-23C illustrate respectively a top view of tunneling and port delivery apparatus 650 equipped with an exemplary vascular access port 652, and axonometric views of a port body 656 before and after integration with implantable tunneler tip 651 to form vascular access port 652 as a unified structure.

As shown, tunneling and port delivery apparatus 650 includes an elongated body 653 comprising of a proximal handheld portion 654 connected to tunneler tip 651 via two opposing narrow extensions 655. Tunneler tip 651 and narrow extensions 655 are connected at respective weakened portions 657 that are configured to break under separating forces greater than a predetermined magnitude. The separating forces may include transverse outward and/or inward force, and/or longitudinal pulling force, relative to median plane MP of apparatus 650. The two narrow extensions 655 enclose a gap therebetween sized for accommodating port body 656 and deployment thereof by integration with tunneler tip 651 to form vascular access port 652 as a unified structure. In some embodiments, port body 656 may be identical, similar or equivalent in function, structure and/or in dimensions, at least in part, but not limited, to one or more of port body 11, port body 21, port body 61, first member (port body) 101, port body 201, port body 301, port body 401, port body 451, and port body 501. Port body 656 coupled with a septum member 665 covering a cavity defined by port body 656, and is coupled at rear end thereof to a catheter connector 666 configured for connecting to a proximal end of a catheter for facilitating fluid communication between the cavity and a lumen of the catheter.

Tunneler tip 651 includes two opposing, optionally symmetric, wing-like structures 658, each one is adjoined at a proximal-lateral portion 659 thereof to respective narrow extension 655 via respective weakened portions 657, and the two wing-like structures 658 are adjoined together with a mutual distal-medial portion 660 (shown in FIG. 22B cut in half along median plane MP for demonstration purposes) configured to allow flexing and/or pivoting thereabout, distinct and/or mutual, of one or the two wing-like structures 658. Each wing-like structure 658 includes an inner surface 661 comprising a distal segment 662 being parallel to median plane MP, and a proximal segment 663 angularly diverging from distal segment 662 laterally from median plane MP. Each wing-like structure 658 encloses an elongated slot 667 sized to accommodate a mating elongated ridge 664 extending along a respective side of port body 656, each elongated slot 667 is continuously opened along most or all length thereof at the respective inner surface 661. Wing-like structures 658 and port body 656 are configured such that, when elongated ridges 664 longitudinally engage in elongated slots 667, and when port body 656 is forced to advance distally from the initial delivery configuration shown in FIG. 23A, and until fully integrating with tunneler tip 651 to form vascular access port 652 as a unified structure, port body 656 gradually forces wing-like structures 658 to deform by pulling proximal segments 663 medially towards median plane MP relative to distal segments 662, thereby generating a separation force sufficient to detach tunneler tip 651 from elongated body 653 of apparatus 650 by breaking weakened portions 657. By expanding and/or reshaping the deformed, now detached, tunneler tip 651, it is configured to improve stabilization and/or anchor port 652 in a subcutaneous void by pressing against surrounding tissues.

Each of the following terms written in singular grammatical form: ‘a’, ‘an’, and ‘the’, as used herein, means ‘at least one’, or ‘one or more’. Use of the phrase ‘one or more’ herein does not alter this intended meaning of ‘a’, ‘an’, or ‘the’. Accordingly, the terms ‘a’, ‘an’, and ‘the’, as used herein, may also refer to, and encompass, a plurality of the stated entity or object, unless otherwise specifically defined or stated herein, or, unless the context clearly dictates otherwise. For example, the phrases: ‘a unit’, ‘a device’, ‘an assembly’, ‘a mechanism’, ‘a component’, ‘an element’, and ‘a step or procedure’, as used herein, may also refer to, and encompass, a plurality of units, a plurality of devices, a plurality of assemblies, a plurality of mechanisms, a plurality of components, a plurality of elements, and, a plurality of steps or procedures, respectively.

Each of the following terms: ‘includes’, ‘including’, ‘has’, ‘having’, ‘comprises’, and ‘comprising’, and, their linguistic/grammatical variants, derivatives, or/and conjugates, as used herein, means ‘including, but not limited to’, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof. Each of these terms is considered equivalent in meaning to the phrase ‘consisting essentially of’.

1. A toggling vascular access port, comprising:
a port body coupled with a septum member covering a cavity defined by the port body; and
at least one port body extension restrictedly movable along an at least one defined route on at least one of two opposing sides of the port body;
the toggling vascular access port is selectively changeable from a delivery configuration to a deployed configuration by moving the at least one port body extension along the at least one defined route wherein the at least one port body extension and the port body are approximated along a median plane of the port body and laterally opposing portions of the at least one port body extension are parted transversely to the median plane, thereby reducing length-to-width ratio of the toggling vascular access port.

2. (canceled)

3. (canceled)

4. The toggling vascular access port of claim 1, wherein the at least one port body extension includes a first arm located right to the median plane and a second arm located left to the median plane.

5. The toggling vascular access port of claim 4, wherein, when in the delivery configuration, each one of the first and second arms includes a wide head portion and a narrow body portion, the head portions and in contact with each other along the median plane and the body portions surround distal portion of the port body from both sides of the median plane.

6. The toggling vascular access port of claim 5, configured to separate between the head portions transversely to the median plane when the toggling vascular access port shifts from the delivery configuration.

7. The toggling vascular access port of claim 4, wherein, when in the deployed configuration, the head portions are in juxtaposition from both sides of the port body with each one of the head portions forming a gap with a front end of the port body, thereby allowing tissue ingrowth therebetween.

8. The toggling vascular access port of claim 1, wherein the port body has an inferior portion and a posterior portion, the posterior portion is connected to the septum member and the inferior portion surrounds the cavity below the septum member and includes a first lateral surface spanning most or all right side of the inferior portion and a second lateral surface spanning most or all left side of the inferior portion, wherein the at least one port body extension comprising a first arm slidably connected to the first lateral surface and restrictedly movable along a first defined route and a second arm slidably connected to the second lateral surface and restrictedly movable along a second defined route.

9-11. (canceled)

12. The toggling vascular access port of claim 8, wherein each one of the right and left sides of the inferior portion and/or of the at least one port body extension includes a rail mechanism along a length of the respective first or second lateral surface, facilitating the first and second defined routes, respectively.

13. The toggling vascular access port of claim 12, wherein each rail mechanism includes a pair of geometrically mating curved elongated ridge and groove longitudinally interengaging with each other, wherein one of the ridge and the groove extend along the respective first or second lateral surface and the other of the ridge and the groove extend along the respective first or second arm.

14. The toggling vascular access port of claim 8, wherein, when in the deployed configuration, the first arm covers the first lateral surface and the second arm covers the second lateral surface.

15. The toggling vascular access port of claim 14, configured such that inner surface of each respective first or second arm is in contact with the respective first or second lateral surface with substantially no gap therebetween.

16. The toggling vascular access port of claim 1, wherein, when changing from the delivery configuration to the deployed configuration, the at least one port body extension rotates around an axis of rotation and slides with an inner surface thereof on at least one of two opposing sides of an inferior portion of the port body.

17. The toggling vascular access port of claim 16, wherein the axis of rotation is located farther than and beyond a closer one of the two opposing sides of the inferior portion relative to the inner surface of the port body extension.

18. The toggling vascular access port of claim 16, wherein the inner surface of the at least one port body extension is configured to cover a front end of the port body and not cover most of the at least one of the two opposing sides of the inferior portion, when the toggling vascular access port is in the delivery configuration.

19. The toggling vascular access port of claim 18, wherein, when in the deployed configuration, the at least one port body extension covers at least most of the two opposing sides of the port body inferior portion.

20-24. (canceled)

25. The toggling vascular access port of claim 1, wherein the at least one port body extension is rotatably and slidably connected to the port body and is configured to rotate around an axis of rotation and slide on the at least one of the two opposing sides of the port body when changing from the delivery configuration to the deployed configuration.

26. The toggling vascular access port of claim 1, further comprising a toggle anchoring mechanism integrated in the port body and/or the at least one port body extension and configured to rotate the at least one port body extension around an axis of rotation and translate the at least one port body extension along the at least one of the opposing sides of the port body when changing from the delivery configuration to the deployed configuration.

27. (canceled)

28. The toggling vascular access port of claim 1, configured such that the at least one defined route is inclined to a longitudinal axis of the port body so as to gradually increase or decrease distance between bottom of the port body and bottom of the at least one port body extension, when changing from the delivery configuration to the deployed configuration.

29. A method for deploying a toggling vascular access port in a body of a subject, the method comprising:
forming a surgical opening through skin tissue layers to a subcutaneous target implantation site in the subject body;
through the surgical opening, inserting the toggling vascular access port in a delivery configuration into the target implantation site, wherein the toggling vascular access port includes a port body and at least one port body extension; and
changing the toggling vascular access port into a deployed configuration by moving the at least one port body extension on at least one of two opposing sides of the port body, thereby approximating the at least one port body extension and the port body along a median plane of the port body and parting laterally opposing portions of the at least one port body extension transversely to the median plane, thereby reducing length-to-width ratio of the toggling vascular access port.

30. (canceled)

31. The method of claim 29, wherein the changing includes pushing the port body distally relative to the at least one port body extension.

32. The method of claim 29, wherein a rear end of the port body comprising a port clamping portion, wherein the inserting includes clamping the port clamping portion with medical forceps and pushing the toggling vascular access port to the target implantation site with the medical forceps.
